SEQUENCE LISTING (22 sequences)

SEQ ID NO: 1 - DNA, 21 bp, Artificial Sequence
  Synthetic; RT-PCR forward primer for Oct4
gtattcagccaaacgaccatc

SEQ ID NO: 2 - DNA, 20 bp, Artificial Sequence
  Synthetic; RT-PCR reverse primer for Oct4
gttcgctttctctttcgggc

SEQ ID NO: 3 - DNA, 21 bp, Artificial Sequence
  Synthetic; RT-PCR forward primer for BF1
actcaaaactcgctgggcaac

SEQ ID NO: 4 - DNA, 21 bp, Artificial Sequence
  Synthetic; RT-PCR reverse primer for BF1
cgtgggggaaaaagtaactgg

SEQ ID NO: 5 - DNA, 23 bp, Artificial Sequence
  Synthetic; RT-PCR forward primer for Nurr1
cgatgccttgtgttcaggcgcag

SEQ ID NO: 6 - DNA, 23 bp, Artificial Sequence
  Synthetic; RT-PCR reverse primer for Nurr1
agcctttgcagccctcacaggtg

SEQ ID NO: 7 - DNA, 18 bp, Artificial Sequence
  Synthetic; RT-PCR forward primer for NeuroD
cagaaccaggacatgccc

SEQ ID NO: 8 - DNA, 19 bp, Artificial Sequence
  Synthetic; RT-PCR reverse primer for NeuroD
atcaaaggaagggctggtg

SEQ ID NO: 9 - DNA, 20 bp, Artificial Sequence
  Synthetic; RT-PCR forward primer for GAPDH
accacagtccatgccatcac

SEQ ID NO: 10 - DNA, 20 bp, Artificial Sequence
  Synthetic; RT-PCR reverse primer for GAPDH
tccaccaccctgttgctgta

SEQ ID NO: 11 - DNA, 2769 bp, Homo sapiens
gagattgtttggtactgttttctctctgggcacctctcatttcggaaggccatcagaggcgcccactactgagcggccccggccgccgcagcagcacccggagccccagtcccggtttccccgcggtgccggagcccggagctcgccgccgcccaggcctcaggaatcgaaggggtggttgctgcttttgctacatgacttgccagcgcccgagcctgcggtccaactgcgctgctgccgtagcgctcagtgccgccgctgccgcccgcgccccccgcgcccagttcggcacccaccggtcgccgccgcccgccgcgccgctgtcccgctcccgcgccgccgccgccatttccccccgacgactgggtgatgctggacatgggagataggaaagaggtgaaaatgatccccaagtcctcgttcagcatcaacagcctggtgcccgaggcggtccagaacgacaaccaccacgcgagccacggccaccacaacagccaccacccccagcaccaccaccaccaccaccaccatcaccaccacccgccgccgcccgccccgcaaccgccgccgccgccgcagcagcagcagccgccgccgccgccgcccccggcaccgcagcccccccagacgcggggcgccccggccgccgacgacgacaagggcccccagcagctgctgctcccgccgccgccaccgccaccaccggccgccgccctggacggggctaaagcggtcgggctgggcggcaagggcgagccgggcggcgggccgggggagctggcgcccgtcgggccggacgagaaggagaagggcgccggcgccgggggggaggagaagaagggggcgggcgagggcggcaaggacggggaggggggcaaggagggcgagaagaagaacggcaagtacgagaagccgccgttcagctacaacgcgctcatcatgatggccatccggcagagccccgagaagcggctcacgctcaacggcatctacgagttcatcatgaagaacttcccttactaccgcgagaacaagcagggctggcagaactccatccgccacaatctgtccctcaacatgtgcttcgtgaaggtgccgcgccactacgacgacccgggcaagggcaactactggatgctggacccgtcgagcgacgacgtgttcatcggcggcaccacgggcaagctgcggcgccgctccaccacctcgcgggccaagctggccttcaagcgcggtgcgcgcctcacctccaccggcctcaccttcatggaccgcgccggctccctctactggcccatgtcgcccttcctgtccctgcaccacccccgcgccagcagcactttgagttacaacggcaccacgtcggcctaccccagccaccccatgccctacagctccgtgttgactcagaactcgctgggcaacaaccactccttctccaccgccaacggcctgagcgtggaccggctggtcaacggggagatcccgtacgccacgcaccacctcacggccgccgcgctagccgcctcggtgccctgcggcctgtcggtgccctgctctgggacctactccctcaacccctgctccgtcaacctgctcgcgggccagaccagttactttttcccccacgtcccgcacccgtcaatgacttcgcagagcagcacgtccatgagcgccagggccacgtcctcctccacgtcgccgcaggccccctcgaccctgccctgtgagtctttaagaccctctttgccaagttttacgacgggactgtctgggggactgtctgattatttcacacatcaaaatcaggggtcttcttccaaccctttaatacattaacatccctgggaccagactgtaagtgaacgttttacacacatttgcattgtaaatgataattaaaaaaataagtccaggtattttttattaagcccccccctcccatttctgtacgtttgttcagtctctagggttgtttattattctaacaaggtgtggagtgtcagcgaggtgcaatgtggggagaatacattgtagaatataaggtttggaagtcaaattatagtagaatgtgtatctaaatagtgactgctttgccatttcattcaaacctgacaagtctatctctaagagccgccagatttccatgtgtgcagtattataagttatcatggaactatatggtggacgcagaccttgagaacaacctaaattatggggagaattttaaaatgttaaactgtaatttgtatttaaaaagcattcgtagtaaaggtgcccaagaaattattttggccatttattgttttgtccttttctttaaagaactgtttttttttcttttgtttacttttagaccaaagattgggttctagaaaatgcacttggtatactaagtattaaaacaaacaaaaaggagagttgtttcagttggcaacactgcccattcaattgaatcagaaggggacaaaattaacgattgccttcagtttgtgttgtgtatattttgatgtatgtggtcactaacaggtcacttttattttttctaaatgtagtgaaatgttaatacctattgtacttataggtaaaccttgcaaatatgtaacctgtgttgcgcaaatgccgcataaatttgagtgattgttaatgttgtcttaaaatttcttgattgtgatactgtggtcatatgcccgtgtttgtcacttacaaaaatgtttactatgaacacacagaaataaaaaataggctaaatccaaaaaaaaaaaaaaa

SEQ ID NO: 12 - DNA, 10883 bp, Homo sapiens
ttccccgtggccgcgccctgctccctcgcggaaggggaagggaacccgggagggcgcctctgagctggagttgctgctattttgggcccagcctggccggtgcgtcaatgcctgcgcccctgcgtcaccgccacccccacccccttccttctccccagctctccaaactctcactttttccctttcgttcgcatggaatcgcttctgcccacggggcgctgacgctaccgagctcatgctaatatgctattcttcgccctcccctcctcatccccctactcacacaccctcacccgccaccacaacccgccccccccacccacttcctggttttattttttttctcgcgcccgctagggcatcgccgagcagcggcggcggcggcggcagcagcagcggcagccgcaacatctgggggaaacttaaggtggtcacgtaggtttccccgaggctgcggcgcaaaagagcccgcggcttcctggggcagccgcagggcatctgatggcaacgcctccccagccgccggcgagctgggggcagcagggagtaggcaaaggcggggctgcagattagggttgagcagcccgggatcccgaatagttccacggagccatcggccggtactgagaacacgagtaggggggcaagttgcaaaagggtcccggctgcctggaaacgagccacaccgagaaaggaggggcggctgggacgctctgcgcgagtagaaaagcgggggcggctccgaactctccctcccaaggcccagccgctggaagccccgcgggagaagccctaggaagccctcccagctccggccgggagacggggagggggcttgccctccggagctgcggcgtttcctttgctagaaagctccctgcgctctcggcgtggctaggggaaatgacttgcaaagcagaagcggcggcgacttggacggccgcgggggtgggacacgctcccacagccagctcgggcccctctcctggctgggctgtaggctcacaccttacgctttgcggagacgctgcccccaatccccacccgcttcgcttttcttgtccagtctttctgggctctaagattgaggctgtgatagaccttgtgtttcaaacgaagatcgatttattcccttaaaactcgtggaaggggtacgagttggtgggcacagaggagtatcgaaagtattaagagtttgcaagaaaggtggggaatcaagagtacctttcttgcagaaggcgcacctgttgcctgctgctcggcttgcattgtctgcgaaaggaaaaggggcgacgcttcactctgacttttggggtttctaaagagctgggtcacggtcactggctaccttttcccttcttatccccccccccccgcaagcaccacgtcctccatcgaacgtgggcactgcatggaaataaggaaacatagaaaaataagccctacccccactcccattccctttcagatgggagtgtggggggtgggggtggggtagagagagtgagagagagagataattagaatgaatatatgccagaagaggaggaggcctgggacaggaaaagggagtaaaaggggatgaaccgggtagggaaatcccacccgaactgcgtgagccctctgagcgtctcgtgtcatgggacatctgtacgctcttccgctaagggggtgacgaaggtgggtgggagaggttagggcgctgcaaggcacatcccattccgtgcggctccaggtgcgtactacctggcccacgccggccttgccttccgccggtgcttttgttgcaccctccccacatgtgagcggccgagccgctgcgcgggcgcaggggcttcggggaaagtgaagtgtcgcgacgctgcgggctgcgcagacctgggagaggtcacacctctttcggaaaaaaaaaaagaaaagaaaaaaaacaccaaaaaccacccaagctggctaccaaggtgaacgcagagcggttcccaccttaaaatcggccctgctcgtgacgtcaggtcggaaatataccaaagcgagcgcgggccaggagtccagggagcgcggcagcgcggcgattgggcggcgggccgctgacgcgcgctgacgcgcggagactttaggtgcatgttggcagcggcagcgcaagccacataaacaaaggcacattggcggccagggccagtccgcccggcggctcgcgcacggctccgcggtcccttttgcctgtccagccggccgcctgtccctgctccctccctccgtgagtgtccgggttcccttcgcccagctctcccacccctacccgaccccggcgcccgggctcccagagggaactgcacttcggcagagttgaatgaatgaagagagacgcggagaactcctaagtgagtagatgcagcccatgcagttcggcccttcttttatgctttttccttcttttgcacgtctcttctttcccacttgtgtgggacaggttctctggaaagtgggagccagaggcttcctagtgagagtgggaccgaaggatggggagtgcgtgcgcgcagttaccggggggcatttgttcgaactccggctttggcactagtggggagttggctctcgacagaggtttccaggctcctcattggtggacgtggaagggagactccacagtttgggagctgaggactagcccgcggaaatgtgcgcaaagtttggctgttagtgaggaattgattgtggcctgtgaacacggagactccaagtcctatgtatacgagggaagctgcccacaaactgacagggagaggaagttcttcagtttatgcgttgcttgggaactgtgtctccgcggctggccagcgcgcgatgtttcccgggtattgttgagtaagggtggtgttggtagccgtgtcctgttactaagttgcctgaaatttctggttttgacatatgctgtccttgggtttgcgaatgtatcagagcgagaatatgaatatgtaaagagtcagttatgaaactgtggagctaccagggggttaatatccaacacaggaatatctctaagggctgtggggttcgagtctcctttctgctttttctgggtaatcttccccccacccccaccaccactatgaagcaagattgtgggggaggggaaagaataaaaagagaggatactggctttcttttttcattagtaataagattgtctgtgctctagaatgtcttccaagtgggaacatctgaaaattactaggacacaagaatgccttgttccagaaaggcagattgtggaaggcattatggggaaggtgttcatcttgctgtgctgggaaacacttctaatattggtgccaataccatataagcagtatgtcccccctctgcaattgacctaagaagctcctggaaaagtagatcccctcttcccaccttgtgaccattaaagccttgtgaccattaaagatgctgaaagacaagttttctggaaaagtgaacatcaatttatctgtagctccaatcccagtgctctgtcaaaagcactttagaagtgcggatgcttccactcaacttgccttctcagtcaaggcctttctaacattttgtaagggggaagattgttttctcattttatattcttgacttctactttcttcccctctaccaaaagaaaaggcaatttcaccacaagaaaaaaaaatgcaagagaaggttccaatgctgtattttcatactctagtcttcatactcaggtcctgaattaacctaagatggaaatgacctctccacctacactgtagcaaaggggccagttcattacatcataaatgttaaatgagttcatggactagctttcctcttgcaggatcttctctctgcaaggatttacacagtgcaatgggtggtattttctgttgtttcaagtcatttcttttatacattcattttaagtgctatgtttggtaaaggcttcccactcatttccaatgagacaaacagggaaggcatggaagggcctgcctggtgagtctacatatgcccagctgaatctctgtcgggaagaaaccctgaagcttcctgtgtctgtatttcagggaggagattggacaggctggactccccattgcttttctaaaaatcttggaaactttgtccttcattgaattacgacactgtccacctttaatttcctcgaaaacgcctgtaactcggctgaaggttagtgcaacttcatttctttcctttactctccagagctccccaaacatcaagaaacaggacaaggcaaaccctgtaacttaaggtttgcccgacccatcgccttcgggaacaactttctcattgtgaaattcaacttcatttctagatggtcatttctagaaagagactgctgaatctgagcttcagagaagaggctcatctgagtgggatgagtgggggggtatgagggagatgtttggaaatacccaggagtgtagaccctcagtagctttttagctctgggtctttatttggttagtctttccacgccctaaactgttgttctgcagcattctctctctcctgcctttcctctcgcgcccctacatgctctctgactgccgcgggctgccggtgtagctccaggtgtacccgagcccgggagaaagtgttcagttgaccaggctgagtgtgttatcaccctgtttcatttccagccatgccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaaaacgccagtctcccgcctctccctcttctcctttaagcaatcgccccctggcaccccggtgtctagttgccagatgcgcttcgacgggcccctgcacgtccccatgaacccggagcccgccggcagccaccacgtggtggacgggcagaccttcgctgtgcccaaccccattcgcaagcccgcgtccatgggcttcccgggcctgcagatcggccacgcgtctcagctgctcgacacgcaggtgccctcaccgccgtcgcggggctccccctccaacgaggggctgtgcgctgtgtgtggggacaacgcggcctgccaacactacggcgtgcgcacctgtgagggctgcaaaggcttctttaaggtgagcaatggcgggagcggagtaggcaggtagggagcccctagtgcccgggacctcggagtgtgccctctgccttggtgccagtagcccagccccagctctcccgggactgcccagctctccggggtccgccgaagctgccctgcaggagaccatgggctgcggcggggacttccgggtgtctgagaaagggaagcagaaagactgggaggccagggtcgcatccccctcgcattcagccgacccggctggcccccgcccgaagttgctggagccggagttggaagagggtcatttgcatgtgctaggagctgtcttccctgttcagaatgaaattggttaggacagagaaccgtgtctgagctaaccaagtggaacagaattccctatggtcaaattaagtgatctctttatttcgccatcctgattgaataatcttatcattttaaatagagaaggtctccaaggaaatgtaaataatatgaatgcccacggatttgtatttactgagcgtctccttctccttctcttggcatataaaacacagcaaggagcggcaaggttagctcaaatgttaacgctatcaattttcttctgttaaatgccctgggggaggaaaaaagaaaagaaagaaagaaaaggaagagaaaaaaataaaatggaattgtgtgtatgtgtttgtttgtggggaggaatcgtagaccccagtcacataacagaaattttctccgagttgcctgattttcaaaagaagaaaaaaaatgttggtctatattgtctccttttgcagcgcacagtgcaaaaaaatgcaaaatacgtgtgtttagcaaataaaaactgcccagtggacaagcgtcgccggaatcgctgtcagtactgccgatttcagaagtgcctggctgttgggatggtcaaagaaggtaggctgaggggagctgccgaccctccagtttgcgcctttaggaaaccactgctcatactccagcatcacgttccacttcccggtgctggggatctccgactccccctcagtatggcctccaggaccctgcagctgcctgcttgcccggccttccctagagaaagccgccaggcccttctctcctttaactatacgacccatttggaggaagacataaaataaccccgcattttttaatgcttctagtcagtgaaggctttacaagcactggggccctcagccgctcagcctggtgccccgcggctgcggccttccccggggagggaccgaggcagcagctgggcctgggctcggaaaagcggcgctaacagggctcttcctttgcagtggttcgcacagacagtttaaaaggccggagaggtcgtttgccctcgaaaccgaagagcccacaggagccctctcccccttcgcccccggtgagtctgatcagtgccctcgtcagggcccatgtcgactccaacccggctatgaccagcctggactattccagggtaagaagctggcgggggggatatcatgtggacaaaccgacagatgggcaggacccctccccacatccgtcattaactctcagattcaacgggggtaaagaaaggcaagcaaggctgtatatgcctcgcagctctggccagggcctcaagattcagatcttcagacaaatccatgtagctgggggcatagacatgaggacaggatggaggaaggaggagagggacacgccacagggtttgaagctgtgtgaattcccactaccccactaccccatcgcccctcctcttccatatacaccagtgcctctaccatgaaatccaggggctgtgcaaactctcccccttcccaatctactttattcccagtcctccatagagatagatgctttaatcctcatccttcctggcactgtgctggggaaggatgtgggggctgtctgggggtcagggaagggaaggagagggtgtaaagatgccagtggggtgggggatcaagtggtcagatccttttactccagctgtgaaaaatatgcgggctttaattggaggaagtatgttgagcaaacctggtagggactgcaattttattaagatttgcaaaagggcgtctcagctcgaggcccactctgggactagcatgaatactaacatgtcaattgttttgtggagataagagtgaacgtttcccagggctggatggcactgtatttagtctgtatggaaatgacaatttacatatttaaagcagcgacctcgtagcaccatccctaattgaattaattgccccggaacatctaatttccttactggtcagagagaggtttaattgttataaaaacctggctcccctattagaaacggggttagcaatttcacgggttatatattttagagaacctcattaagtgctttttaaaatgaaattccagttccaggcgaaccctgactatcaaatgagtggagatgacacccagcatatccagcaattctatgatctcctgactggctccatggagatcatccggggctgggcagagaagatccctggcttcgcagacctgcccaaagccgaccaagacctgctttttgaatcagctttcttagaactgtttgtccttcgattagcatacaggtaataagggagggaggagacaatccagggaggctgtgagagaaatcaagaaaggaaaagaaagggaggaagggaaaccagagggtggggtagagaaaaagacagaataggaaatggaagtcggagaaaggaagaaaaagaaagaaaacaaaaaaagacgagaagaagcgagcccagaagccttggatgaatggaatggaggtgggatagggggcgttcttgattgttatgaaattaaaccctttcaaggtccactggatctacattttaattaactcttcagtaattaggtgactcttaaatccctcatttattgctcttcaagtaattagttgtttagcttttctctctctctttttctcccctctctctctttggtattaattgcaggtccaacccagtggagggtaaactcatcttttgcaatggggtggtcttgcacaggttgcaatgcgttcgtggctttggggaatggattgattccattgttgaattctcctccaacttgcagaatatgaacatcgacatttctgccttctcctgcattgctgccctggctatggtcacaggtcagtactgcaggcgcagggcgcttcccctccagaactgcctagcaggatttgtcctgagtttcccttgtcacagaattctccttggttttgccaactagctaactgtcttgtacattcttcttttgtttctgattatgttttctgcagagagacacgggctcaaggaacccaagagagtggaagaactgcaaaacaagattgtaaattgtctcaaagaccacgtgactttcaacaatggggggttgaaccgccccaattatttgtccaaactgttggggaagctcccagaacttcgtaccctttgcacacaggggctacagcgcattttctacctgaaattggaagacttggtgccaccgccagcaataattgacaaacttttcctggacactttacctttctaagacctcctcccaagcacttcaaaggaactggaatgataatggaaactgtcaagagggggcaagtcacatgggcagagatagccgtgtgagcagtctcagctcaagctgccccccatttctgtaaccctcctagcccccttgatccctaaagaaaacaaacaaacaaacaaaaactgttgctatttcctaacctgcaggcagaacctgaaagggcattttggctccggggcatcctggatttagaacatggactacacacaatacagtggtataaactttttattctcagtttaaaaatcagtttgttgttcagaagaaagattgctataaggtataatgggaaatgtttggccatgcttggttgttgcagttcagacaaatgtaacacacacacacatacacacacacacacacacagagacacatcttaaggggacccacaagtattgccctttaacaagacttcaaagttttctgctgtaaagaaagctgtaatatatagtaaaactaaatgttgcgtgggtggcatgagttgaagaaggcaaaggcttgtaaatttacccaatgcagtttggctttttaaattattttgtgcctatttatgaataaatattacaaattctaaaagataagtgtgtttgcaaaaaaaaagaaaataaatacataaaaaagggacaagcatgttgattctaggttgaaaatgttataggcacttgctacttcagtaatgtctatattatataaatagtatttcagacactatgtagtctgttagattttataaagattggtagttatctgagcttaaacattttctcaattgtaaaataggtgggcacaagtattacacatcagaaaatcctgccaaaagggccacatagtgtttgtaacaccgtccaacattccttgtttgtaagtgttgtatgtaccgttgatgttgataaaaagaaagtttatatcttgattattttgttgtctaaagctaaacaaaacttgcatgcagcagcttttgactgtttccagagtgcttataatatacataactccctggaaataactgagcactttgaattttttttatgtctaaaattgtcagttaatttattattttgtttgagtaagaattttaatattgccatattctgtagtatttttctttgtatatttctagtatggcacatgatatgagtcactgcctttttttctatggtgtatgacagttagagatgctgattttttttctgataaattctttctttgagaaagacaattttaatgtttacaacaataaaccatgtaaatgatacagaattttgtcttctttttgggtcagaaaaaataatgactaaacagcaacataacatacacagttggagattaatttgggataggtcacttattcaatatctgaattttaaacagcctaaatctattctggcaaaagaaatcaggtgaaccagaataattatccagataattcagtaaaatattttttcttaaccagaattttccagtctggtgaccaatatttattaggatcaatttagaatatctcaattcttctgttaactttgcaagtaagtgtagctttcaggagagaagttctggagacccaaaagcttttgaacttgagcaaggtcagagtctagtcaagtacttgtcttgcacagtgattaggaagtatctttttaagactcaagaagtcaacatggatttgaggagtgttgtatgtaatctccgattttccaatttctcaattt

SEQ ID NO: 13 - DNA, 1676 bp, Homo sapiens
acatcgattaactttttctcagaggcattcattttgtaatgggcaggtacttttcgcaagcatttgtacaggtttagggagtggaagctgaaggcgatctttcttttgatatagcgtttttctgcttttctttctgtttgcctctcccttgttgaatgtaggaaatcgaaacatgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatctcgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccgcacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattagaggcacgccagtttcaccatttccgggaaacgaacccactgtgcttacagtgactgtcgtgtttacaaaaggcagccctttggtactactgctgcaaagtgcaaatactccaagcttcaagtgatatatgtatttattgtcattactgcctttggaagaaacaggggatcaaagttcctgttcaccttatgtattattttctatagactcttctattttaaaaaataaaaaaatacagtaaagtttaaaaaatacaccacgaatttggtgtggctgtattcagatcgtattaattatctgatcgggataacaaaatcacaagcaataattaggatctatgcaatttttaaactagtaatgggccaattaaaatatatataaatatatatttcaaccagcattttactacttgttacctcccatgctgaattat

SEQ ID NO: 14 - DNA, 1189 bp, Homo sapiens
ccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaa

SEQ ID NO: 15 - DNA, 2114 bp, Homo sapiens
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcctatccagtcaatctcatggagggtggagtatggttggagcctaatcagcgaggtttctttttttttttttcctattggatcttcctggagaaaatacttttttttttttttttgagacggagtcttgctctgtcgcccaggctggagtgcagtggcgcggtcttggctcactgcaagctccgcctcccgggttcacgccattctcctgcctcagcctcccgagcagctgggactacaggcgcccgccacctcgcccggctaatattttgtatttttagtagagacagggtttcactgtgttagccaggatggtctcgatctcctgaccttgtgatccgcccgcctcggcctccctaacagctgggattacaggcgtgagccaccgcgccctgcctagaaaagacattttaataaccttggctgctaaggacaacattgatagaagccgtctctggctatagataagtagatctaatactagtttggatatctttagggtttagaatctaacctcaagaataagaaatacaagtacgaattggtgatgaagatgtattcgtattgtttgggattgggaggctttgcttatttttttaaaactattgaggtaaagggttaagctgtaacatacttaattgatttcttaccgtttttggctctgttttgctatatcccctaatttgttggttgtgctaatctttgtagaaagaggtcttgtatttgctgcatcgtaatgacatgagtactactttagttggtttaagttcaaatgaatgaaacaaatatttttcctttagttgattttaccctgatttcaccgagtgtttcgatgagtaaatatacagcttaaacat

SEQ ID NO: 16 - DNA, 1698 bp, Homo sapiens
cagaggtcaggcttcgctaatgggccagtgaggagcggtggaggcgaggccggcgccgcacacacacattaacacacttgagccatcaccaatcagcataggaatctgagaattgctctcacacaccaacccagcaacatccgtggagaaaactctcaccagcaactcctttaaaacaccgtcatttcaaaccattgtggtcttcaagcaacaacagcagcacaaaaaaccccaaccaaacaaaactcttgacagaagctgtgacaaccagaaaggatgcctcataaagggggaagactttaactaggggcgcgcagatgtgtgaggccttttattgtgagagtggacagacatccgagatttcagagccccatattcgagccccgtggaatcccgcggcccccagccagagccagcatgcagaacagtcacagcggagtgaatcagctcggtggtgtctttgtcaacgggcggccactgccggactccacccggcagaagattgtagagctagctcacagcggggcccggccgtgcgacatttcccgaattctgcaggtgtccaacggatgtgtgagtaaaattctgggcaggtattacgagactggctccatcagacccagggcaatcggtggtagtaaaccgagagtagcgactccagaagttgtaagcaaaatagcccagtataagcgggagtgcccgtccatctttgcttgggaaatccgagacagattactgtccgagggggtctgtaccaacgataacataccaagcgtgtcatcaataaacagagttcttcgcaacctggctagcgaaaagcaacagatgggcgcagacggcatgtatgataaactaaggatgttgaacgggcagaccggaagctggggcacccgccctggttggtatccggggacttcggtgccagggcaacctacgcaagatggctgccagcaacaggaaggagggggagagaataccaactccatcagttccaacggagaagattcagatgaggctcaaatgcgacttcagctgaagcggaagctgcaaagaaatagaacatcctttacccaagagcaaattgaggccctggagaaagagtttgagagaacccattatccagatgtgtttgcccgagaaagactagcagccaaaatagatctacctgaagcaagaatacaggtatggttttctaatcgaagggccaaatggagaagagaagaaaaactgaggaatcagagaagacaggccagcaacacacctagtcatattcctatcagcagtagtttcagcaccagtgtctaccaaccaattccacaacccaccacaccggtttcctccttcacatctggctccatgttgggccgaacagacacagccctcacaaacacctacagcgctctgccgcctatgcccagcttcaccatggcaaataacctgcctatgcaacccccagtccccagccagacctcctcatactcctgcatgctgcccaccagcccttcggtgaatgggcggagttatgatacctacacccccccacatatgcagacacacatgaacagtcagccaatgggcacctcgggcaccacttcaacaggactcatttcccctggtgtgtcagttccagttcaagttcccggaagtgaacctgatatgtctcaatactggccaagattacagtaaaaaaaaaaaaaa

SEQ ID NO: 17 - DNA, 4091 bp, Homo sapiens
  misc_feature (2313)..(2313): n is a, c, g, or t
ccggccgtctatgctccaggccctctcctcgcggtgccggtgaacccgccagccgccccgatgtacagcatgatgatggagaccgacctgcactcgcccggcggcgcccaggcccccacgaacctctcgggccccgccggggcgggcggcggcgggggcggaggcgggggcggcggcggcggcgggggcgccaaggccaaccaggaccgggtcaaacggcccatgaacgccttcatggtgtggtcccgcgggcagcggcgcaagatggcccaggagaaccccaagatgcacaactcggagatcagcaagcgcctgggggccgagtggaaggtcatgtccgaggccgagaagcggccgttcatcgacgaggccaagcggctgcgcgcgctgcacatgaaggagcacccggattacaagtaccggccgcgccgcaagaccaagacgctgctcaagaaggacaagtactcgctggccggcgggctcctggcggccggcgcgggtggcggcggcgcggctgtggccatgggcgtgggcgtgggcgtgggcgcggcgcccgtgggccagcgcctggagagcccaggcggcgcggcgggcggcgcgtacgcgcacgtcaacggctgggccaacggcgcctaccccggctcggtggcggccgcggcggccgccgcggccatgatgcaggaggcgcagctggcctacgggcagcaccccggcgcgggcggcgcgcacccgcaccgcaccccggcgcacccgcacccgcaccacccgcacgcgcacccgcacaacccgcagcccatgcaccgctacgacatgggcgcgctgcagtacagccccatctccaactcgcagggctacatgagcgcgtcgccctcgggctacggcggcctcccctacggcgccgcggccgccgccgccgccgcgcaccagaactcggccgtggcggcggcggcggcggcggcggccgcgtcgtcgggcgccctgggcgcgctgggctctctggtgaagtcggagcccagcggcagcccgcccgccccagcgcactcgcgggcgccgtgccccggggacctgcgcgagatgatcagcatgtacttgcccgccggcgaggggggcgacccggcggcggcagcagcggccgcggcgcagagccggctgcactcgctgccgcagcactaccagggcgcgggcgcgggcgtgaacggcacggtgcccctgacgcacatctagcgccttcgggacgccggggactctgcggcggcgacccacgagctcgcggcccgcgcccggctcccgccccgccccggcgcggcgtggcttttgtatcagacgttcccacattcttgtcaaaaggaaaatactggagacgaacgccgggtgacgcgtgtcccccactcaccttccccggagaccctggcgaccgccgggcgctgacaccagacttggtttagactgaacttcggtgttttcttgagacttttgtacagtatttatcacctacggaggaagcggaagcgttttctttgctcgaggggacaaaaaagtcaaaacgaggcgagaggcgaagcccacttttgtataccggccggcgcgctcactttcctccgcgttgcttccggacggcgccgaccgccggagcccaagtgacgcggagctcgtcgcatttgttataaatgtagtaaggcaggtccaagcacttacaagttttttgtagttgttaccgctcttttgggttggtttgttaatttatacaaagagattaccaccaccaccccctccttcagacggcggagttatattctgggttttgtaaaactttatgtatctgagcatttccatttttttttttgggttttgtattatttcttgtaaatgcattgtgaaaaattttattttcggcgttgcaatgcggggaggagaagtcagattatgtacatagttttctaaaaagcctttcttctaaaaacgaaaaaagacccccacccaaaatgtttcgagtcaacaaatttaagagacagagcccattttctccataaatttgtaacatgcctatttttatgtgcatgttttatgagttcaaaatgcaatgagggaaatctgacagggaaattatctgtatgaactaaaagtaagggaacccggggaatgggaggacaggatttttcaaggaacctttttcaatgaaagagaaggaagttaaaacctataggttattttgtagagctgagtgttaatacgggccgagaaataaaagtatcttctgctccggctgtttcactgcggacggctggggctgctgcgcgttaccttgctgcaacngggcgccttccacctggctgggggtctgcgccacagtttggtccagangwgggaggaggaagggaagaccccagtggtgggaccctggaccaggccatggatgaaggacaaagaccagggcaggtcacgggtttcccaattccccagcaattaagatttcgagcagaatttatctaaatgtgtttcaaggaaacacaatcgctgaaccaaaacgtactgcagccganccccctccgtccatcctctgcccctccccctggcttctttctcttgggaaaacgggcaaaataattgtgctggattctcacacacacagaaatatcgaccatcaccctcccccgcgtgaactgggatgcaagttgctaaccgatgtgaacgcaaaatgccttgttcattattcctgacgagatcttgaggttgtttgatgctttaaattttttaattatattattttctaggtgtttattggtacattgcagttttttttttgaaatttaaaaatttctgtaaaactttgtcttcaagtaatctgacagcattaaatattgcatttaaaaattatactgtagcaaatacatttaaaaattaatcacaacgttaagatgaaattatatttttggaaaaaaaaaacacttgaagcccagatggaaatacgtttatttcagcagccttaggtttcccctcgctttctcaacacccttccttgtcctggagtatggactgtccgtccaaaagtgagcctatgctataagtttaatgagaaccgaattcagcctgcattcgagaatagctttaagtataatgctgatctgacaattgacgtgtaatttgggaagtcattttgataattttgcttaaaccactcattcgttaaagtgattacaaaaaagttcaagaatgatgtccactgctttctaacaagataataaaccccccccctcttttctttttctttatttttatttcttttagctatttgatcctttctgaagcagttgtttctggaagagtctgtgcgcccatggatggctgagcaccactacgacttagtccgggataagggcctccccagtcctctccgggagatgatttgggaaattttataatgcttgttctgttaactcaccgggaccttgagggtccaatgggaccttgagggttttctctgaaatatacaaacttaaaggactctctctgaggttctttgactgacgtccactctcagtctggcccctgtgctcccctgtgtgtaccctggagtttctgtgtccaattgttggcatctaggtcttggctcaagattaggatgtgggccccactttagaggcacagactatgaaaagctgagttagtgcgcccgggacgccaggcaagcagcttttacagtttggcatcttattgcaggtgcttcgtgcacagtcagctgaaatagccaatgccaggtgctccaaccaccttatttccttgttttgttgattagaacaacacagaaaaaagcaaatataaatttttaatgactccatttaaaaatatcacagggtgggggcaaggaaattagctgagattcatctcaggattgagattctatccccccttccccgcccccagcagtgtcgctccaattcaaattagtggagaaaagattacagtaggccctgagccgactgtgaattcggtgcttggccaaggtaacactcatcgtattcacggagraaatactatatgatgatagttattatattatatgacgacttcattcacttcccaaatcacagggt

SEQ ID NO: 18 - DNA, 5591 bp, Homo sapiens
gctactcccaccccgccccgccccgtcattgtccccgtcggtctcttttctcttccgtcctaaaagctctgcgagccgctcccttctcccggtgccccgcgtctgtccatcctcagtgggtcagacgagcaggatggagggctgcatgggggaggagtcgtttcagatgtgggagctcaatcggcgcctggaggcctacctggcccgggtcaaggcgctggaggagcagaatgagctgctcagcgcggagctcggggggctccgggcacaatccgcggacacctcctggcgggcgcatgccgacgacgagctggcggccctgcgggccctcgttgaccaacgctggcgggagaagcacgcggccgaggtggcgcgcgacaacctggctgaagagctggagggcgtggcaggccgatgccagcagctgcggctggcccgggagcggacgacggaggaggtagcccgcaaccggcgcgccgtcgaggcagagaaatgcgcccgggcctggctgagtagccaggtggcagagctggagcgcgagctagaggctctacgcgtggcgcacgaggaggagcgcgtcggcctgaacgcgcaggctgcctgtgccccccgctgccccgcgccgccccgcgggcctcccgcgccggccccggaggtagaggagctggcaaggcgactgggcgaggcgtggcgcggggcagtgcgcggctaccaggagcgcgtggcacacatggagacgtcgctgggccaggcccgcgagcggctgggccgggcggtgcagggtgcccgcgagggccgcctggagctgcagcagctccaggctgagcgcggaggcctcctggagcgcagggcagcgttggaacagaggttggagggccgctggcaggagcggctgcgggctactgaaaagttccagctggctgtggaggccctggagcaggagaaacagggcctacagagccagatcgctcaggtcctggaaggtcggcagcagctggcgcacctcaagatgtccctcagcctggaggtggccacgtacaggaccctcctggaggctgagaactcccggctgcaaacacctggcggtggctccaagacttccctcagctttcaggaccccaagctggagctgcaattccctaggaccccagagggccggcgtcttggatctttgctcccagtcctgagcccaacttccctcccctcacccttgcctgctacccttgagacacctgtgccagcctttcttaagaaccaagaattcctccaggcccgtacccctaccttggccagcacccccatcccccccacacctcaggcaccctctcctgctgtagatgcagagatcagagcccaggatgctcctctctctctgctccagacacagggtgggaggaaacaggctccagagcccctgcgggctgaagccagggtggccattcctgccagcgtcctgcctggaccagaggagcctgggggccagcggcaagaggccagtacaggccagtccccagaggaccatgcctccttggcaccacccctcagccctgaccactccagtttagaggctaaggatggagaatccggtgggtctagagtgttcagcatatgccgaggggaaggtgaagggcaaatctgggggttggtagagaaagaaacagccatagagggcaaagtggtaagcagcttgcagcaggaaatatgggaagaagaggatctaaacaggaaggaaatccaggactcccaggttcctttggaaaaagaaaccctgaagtctctgggagaggagattcaagagtcactgaagactctggaaaaccagagccatgagacactagaaagggagaatcaagaatgtccgaggtctttagaagaagacttagaaacactaaaaagtctagaaaaggaaaataaagagctattaaaggatgtggaggtagtgagacctctagaaaaagaggctgtaggccaacttaagcctacaggaaaagaggacacacagacattgcaatccctgcaaaaggagaatcaagaactaatgaaatctcttgaaggtaatctagagacatttttatttccaggaacggaaaatcaagaattagtaagttctctgcaagagaacttagagtcattgacagctctggaaaaggagaatcaagagccactgagatctccagaagtaggggatgaggaggcactgagacctctgacaaaggagaatcaggaacccctgaggtctcttgaagatgagaacaaagaggcctttagatctctagaaaaagagaaccaggagccactgaagactctagaagaagaggaccagagtattgtgagacctctagaaacagagaatcacaaatcactgaggtctttagaagaacaggaccaagagacattgagaactcttgaaaaagagactcaacagcgacggaggtctctaggggaacaggatcagatgacattaagacccccagaaaaagtggatctagaaccactgaagtctcttgaccaggagatagctagacctcttgaaaatgagaatcaagagttcttaaagtcactcaaagaagagagcgtagaggcagtaaaatctttagaaacagagatcctagaatcactgaagtctgcgggacaagagaacctggaaacactgaaatctccagaaactcaagcaccactgtggactccagaagaaataaatcagggggcaatgaatcctctagaaaaggaaattcaagaaccactggagtctgtggaagtgaaccaagagacattcagactcctggaagaggagaatcaggaatcattgagatctctgggagcatggaacctggagaatttgagatctccagaggaggtagacaaggaaagtcaaaggaatctggaagaggaagagaacctgggaaagggagagtaccaagagtcactgaggtctctggaggaggagggacaggagctgccgcagtctgcagatgtgcagaggtgggaagatacggtggagaaggaccaagaactggctcaggaaagccctcctgggatggctggagtggaaaatgaggatgaggcagagctgaatctgagggagcaggatggcttcactgggaaggaggaggtggtagagcagggagagctgaatgccacagaggaggtctggatcccaggcgaggggcacccagagagccctgagcccaaagagcagagaggcctggttgagggagccagtgtgaagggaggggctgagggcctccaggaccctgaagggcaatcacaacaggtgggggccccaggcctccaggctccccaggggctgccagaggcgatagagcccctggtggaagatgatgtggccccagggggtgaccaagcctccccagaggtcatgttggggtcagagcctgccatgggtgagtctgctgcgggagctgagccaggcccggggcagggggtgggagggctgggggacccaggccatctgaccagggaagaggtgatggaaccacccctggaagaggagagtttggaggcaaagagggttcagggcttggaagggcctagaaaggacctagaggaggcaggtggtctggggacagagttctccgagctgcctgggaagagcagagacccttgggagcctcccagggagggtagggaggagtcagaggctgaggcccccaggggagcagaggaggcgttccctgctgagaccctgggccacactggaagtgatgccccttcaccttggcctctggggtcagaggaagctgaggaggatgtaccaccagtgctggtctcccccagcccaacgtacaccccgatcctggaagatgcccctgggcctcagcctcaggctgaagggagtcaggaggctagctggggggtgcaggggagggctgaagccctggggaaagtagagagcgagcaggaggagttgggttctggggagatccccgagggcccccaggaggaaggggaggagagcagagaagagagcgaggaggatgagctcggggagacccttccagactccactcccctgggcttctacctcaggtcccccacctcccccaggtgggaccccactggagagcagaggccaccccctcaaggggagactggaaaggagggctgggatcctgctgtcctggcttccgagggccttgaggccccaccctcagaaaaggaggagggggaggagggagaagaggagtgtggccgtgactctgacctgtcagaagaatttgaggacctggggactgaggcaccttttcttcctggggtccctggggaggtggcagaacctctgggccaggtgccccagctgctactggatcctgcagcctgggatcgagatggggagtccgatgggtttgcagatgaggaagaaagtggggaggagggagaggaggatcaggaggaggggagggagccaggggctgggcggtgggggccagggtcttctgttggcagcctccaggccctgagtagctcccagagaggggaattcctggagtctgattctgtgagtgtcagtgtcccctgggatgacagcttgaggggtgcagtggctggtgcccccaagactgccctggaaacggagtcccaggacagtgctgagccttctggctcagaggaagagtctgaccctgtttccttggagagggaggacaaagtccctggccctctagagatccccagtgggatggaggatgcaggcccaggggcagacatcattggtgttaatggccagggtcccaacttggaggggaagtcacagcatgtgaatgggggagtgatgaacgggctggagcagtctgaggaagtggggcaaggaatgccgctagtctctgagggagaccgagggagcccctttcaggaggaggaggggagtgctctgaagacctcttgggcaggggctcctgttcacctgggccagggtcagttcctgaagttcactcagagggaaggagatagagagtcctggtcctcaggggaggactaggaaaagaccatctgcccggcactggggacttaggggtgcggggaggggaaggacgcctccaagcccgctccctgctcaggagcagcactcttaacttacgatctcttgacatatggtttctggctgagaggcctggcccgctaaggtgaaaaggggtgtggcaaaggagcctactccaagaatggaggctgtaggaatataacctcccaccctgcaaagggaatctcttgcctgctccatctcataggctaagtcagctgaatcccgatagtactaggtccccttccctccgcatcccgtcagctggaaaaggcctgtggcccagaggcttctccaaagggagggtgacatgctggcttttgtgcccaagctcaccagccctgcgccacctcactgcagtagtgcaccatctcactgcagtagcacgccctcctgggccgtctggcctgtggctaatggaggtgacggcactcccatgtgctgactccccccatccctgccacgctgtggccctgcctggctagtccctgcctgaataaagtaatgcctccgcttcaaaaaaaaaaaaaaaaaaaaaaaaaaaaaaaaaaaa

SEQ ID NO: 19 - DNA, 1736 bp, Homo sapiens
cgaccctcagcagccagcccggcccgcccgcgcccgtccgcagccgcccgccagacgcgcccagtatgagggagatcgtgcacatccaggccggccagtgcggcaaccagatcggggccaagttctgggaagtcatcagtgatgagcatggcatcgaccccagcggcaactacgtgggcgactcggacttgcagctggagcggatcagcgtctactacaacgaggcctcttctcacaagtacgtgcctcgagccattctggtggacctggaacccggaaccatggacagtgtccgctcaggggcctttggacatctcttcaggcctgacaatttcatctttggtcagagtggggccggcaacaactgggccaagggtcactacacggagggggcggagctggtggattcggtcctggatgtggtgcggaaggagtgtgaaaactgcgactgcctgcagggcttccagctgacccactcgctggggggcggcacgggctccggcatgggcacgttgctcatcagcaaggtgcgtgaggagtatcccgaccgcatcatgaacaccttcagcgtcgtgccctcacccaaggtgtcagacacggtggtggagccctacaacgccacgctgtccatccaccagctggtggagaacacggatgagacctactgcatcgacaacgaggcgctctacgacatctgcttccgcaccctcaagctggccacgcccacctacggggacctcaaccacctggtatcggccaccatgagcggagtcaccacctccttgcgcttcccgggccagctcaacgctgacctgcgcaagctggccgtcaacatggtgcccttcccgcgcctgcacttcttcatgcccggcttcgcccccctcacagcccggggcagccagcagtaccgggccctgaccgtgcccgagctcacccagcagatgttcgatgccaagaacatgatggccgcctgcgacccgcgccacggccgctacctgacggtggccaccgtgttccggggccgcatgtccatgaaggaggtggacgagcagatgctggccatccagagcaagaacagcagctacttcgtggagtggatccccaacaacgtgaaggtggccgtgtgtgacatcccgccccgcggcctcaagatgtcctccaccttcatcgggaacagcacggccatccaggagctgttcaagcgcatctccgagcagttcacggccatgttccggcgcaaggccttcctgcactggtacacgggcgagggcatggacgagatggagttcaccgaggccgagagcaacatgaacgacctggtgtccgagtaccagcagtaccaggacgccacggccgaggaagagggcgagatgtacgaagacgacgaggaggagtcggaggcccagggccccaagtgaagctgctcgcagctggagtgagaggcaggtggcggccggggccgaagccagcagtgtctaaacccccggagccatcttgctgccgacaccctgctttcccctcgccctagggctcccttgccgccctcctgcagtatttatggcctcgtcctccccacctaggccacgtgtgagctgctcctgtctctgtcttattgcagctccaggcctgacgttttacggttttgttttttactggtttgtgtttatattttcggggatacttaataaatctattgctgtcagatacccttaaaaaaaaaaaaaaaaaaaaaaaaa

SEQ ID NO: 20 - DNA, 8305 bp, Homo sapiens
gcgcccaggagcagagccgcgctcgctccactcagctcccagctcccaggactccgctggctcctcgcaagtcctgccgcccagcccgccgggatgcagtccgggccgcggcccccacttccagcccccggcctggccttggctttgaccctgactatgttggccagacttgcatccgcggcttccttcttcggtgagaaccacctggaggtgcctgtggccacggctctgaccgacatagacctgcagctgcagttctccacgtcccagcccgaagccctccttctcctggcagcaggcccagctgaccacctcctgctgcagctctactctggacgcctgcaggtcagacttgttctgggccaggaggagctgaggctgcagactccagcagagacgctgctgagtgactccatcccccacactgtggtgctgactgtcgtagagggctgggccacgttgtcagtcgatgggtttctgaacgcctcctcagcagtcccaggagcccccctagaggtcccctatgggctctttgttgggggcactgggacccttggcctgccctacctgaggggaaccagccgacccctgaggggttgcctccatgcagccaccctcaatggccgcagcctcctccggcctctgacccccgatgtgcatgagggctgtgctgaagagttttctgccagtgatgatgtggccctgggcttctctgggccccactctctggctgccttccctgcctggggcactcaggacgaaggaaccctagagtttacactcaccacacagagccggcaggcacccttggccttccaggcagggggccggcgtggggacttcatctatgtggacatatttgagggccacctgcgggccgtggtggagaagggccagggtaccgtattgctccacaacagtgtgcctgtggccgatgggcagccccatgaggtcagtgtccacatcaatgctcaccggctggaaatctccgtggaccagtaccctacgcatacttcgaaccgaggagtcctcagctacctggagccacggggcagtctccttctcggggggctggatgcagaggcctctcgtcacctccaggaacaccgcctgggcctgacaccagaggccaccaatgcctccctgctgggctgcatggaagacctcagtgtcaatggccagaggcgggggctgcgggaagctttgctgacgcgcaacatggcagccggctgcaggctggaggaggaggagtatgaggacgatgcctatggacattatgaagctttctccaccctggcccctgaggcttggccagccatggagctgcctgagccatgcgtgcctgagccagggctgcctcctgtctttgccaatttcacccagctgctgactatcagcccactggtggtggccgaggggggcacagcctggcttgagtggaggcatgtgcagcccacgctggacctgatggaggctgagctgcgcaaatcccaggtgctgttcagcgtgacccgaggggcacgccatggcgagctcgagctggacatcccgggagcccaggcacgaaaaatgttcaccctcctggacgtggtgaaccgcaaggcccgcttcatccacgatggctctgaggacacctccgaccagctggtgctggaggtgtcggtgacggctcgggtgcccatgccctcatgccttcggaggggccaaacatacctcctgcccatccaggtcaaccctgtcaatgacccaccccacatcatcttcccacatggcagcctcatggtgatcctggaacacacgcagaagccgctggggcctgaggttttccaggcctatgacccggactctgcctgtgagggcctcaccttccaggtccttggcacctcctctggcctccccgtggagcgccgagaccagcctggggagccggcgaccgagttctcctgccgggagttggaggccggcagcctagtctatgtccaccgcggtggtcctgcacaggacttgacgttccgggtcagcgatggactgcaggccagccccccggccacgctgaaggtggtggccatccggccggccatacagatccaccgcagcacagggttgcgactggcccaaggctctgccatgcccatcttgcccgccaacctgtcggtggagaccaatgccgtggggcaggatgtgagcgtgctgttccgcgtcactggggccctgcagtttggggagctgcagaagcagggggcaggtggggtggagggtgctgagtggtgggccacacaggcgttccaccagcgggatgtggagcagggccgcgtgaggtacctgagcactgacccacagcaccacgcttacgacaccgtggagaacctggccctggaggtgcaggtgggccaggagatcctgagcaatctgtccttcccagtgaccatccagagagccactgtgtggatgctgcggctggagccactgcacactcagaacacccagcaggagaccctcaccacagcccacctggaggccaccctggaggaggcaggcccaagccccccaaccttccattatgaggtggttcaggctcccaggaaaggcaaccttcaactacagggcacaaggctgtcagatggccagggcttcacccaggatgacatacaggctggccgggtgacctatggggccacagcacgtgcctcagaggcagtcgaggacaccttccgtttccgtgtcacagctccaccatatttctccccactctataccttccccatccacattggtggtgacccagatgcgcctgtcctcaccaatgtcctcctcgtggtgcctgagggtggtgagggtgtcctctctgctgaccacctctttgtcaagagtctcaacagtgccagctacctctatgaggtcatggagcggccccgccatgggaggttggcttggcgtgggacacaggacaagaccactatggtgacatccttcaccaatgaagacctgttgcgtggccggctggtctaccagcatgatgactccgagaccacagaagatgatatcccatttgttgctacccgccagggcgagagcagtggtgacatggcctgggaggaggtacggggtgtcttccgagtggccatccagcccgtgaatgaccacgcccctgtgcagaccatcagccggatcttccatgtggcccggggtgggcggcggctgctgactacagacgacgtggccttcagcgatgctgactcgggctttgctgacgcccagctggtgcttacccgcaaggacctcctctttggcagtatcgtggccgtagatgagcccacgcggcccatctaccgcttcacccaggaggacctcaggaagaggcgagtactgttcgtgcactcaggggctgaccgtggctggatccagctgcaggtgtccgacgggcaacaccaggccactgcgctgctggaggtgcaggcctcggaaccctacctccgtgtggccaacggctccagccttgtggtccctcaaggaggccagggcaccatcgacacggccgtgctccacctggacaccaacctcgacatccgcagtggggatgaggtccactaccacgtcacagctggccctcgctggggacagctagtccgggctggtcagccagccacagccttctcccagcaggacctgctggatggggccgttctctatagccacaatggcagcctcagcccccgcgacaccatggccttctccgtggaagcagggccagtgcacacggatgccaccctacaagtgaccattgccctagagggcccactggccccactgaagctggtccggcacaagaagatctacgtcttccagggagaggcagctgagatcagaagggaccagctggaggcagcccaggaggcagtgccacctgcagacatcgtattctcagtgaagagcccaccgagtgccggctacctggtgatggtgtcgcgtggcgccttggcagatgagccacccagcctggaccctgtgcagagcttctcccaggaggcagtggacacaggcagggtcctgtacctgcactcccgccctgaggcctggagcgatgccttctcgctggatgtggcctcaggcctgggtgctcccctcgagggcgtccttgtggagctggaggtgctgcccgctgccatcccactagaggcgcaaaacttcagcgtccctgagggtggcagcctcaccctggcccctccactgctccgtgtctccgggccctacttccccactctcctgggcctcagcctgcaggtgctggagccaccccagcatggagccctgcagaaggaggacggacctcaagccaggaccctcagcgccttctcctggagaatggtggaagagcagctgatccgctacgtgcatgacgggagcgagacactgacagacagttttgtcctgatggctaatgcctccgagatggatcgccagagccatcctgtggccttcactgtcactgtcctgcctgtcaatgaccaaccccccatcctcactacaaacacaggcctgcagatgtgggagggggccactgcgcccatccctgcggaggctctgaggagcacggacggcgactctgggtctgaggatctggtctacaccatcgagcagcccagcaacgggcgggtagtgctgcggggggcgccgggcactgaggtgcgcagcttcacgcaggcccagctggacggcgggctcgtgctgttctcacacagaggaaccctggatggaggcttccgcttccgcctctctgacggcgagcacacttcccccggacacttcttccgagtgacggcccagaagcaagtgctcctctcgctgaagggcagccagacactgactgtctgcccagggtccgtccagccactcagcagtcagaccctcagggccagctccagcgcaggcactgacccccagctcctgctctaccgtgtggtgcggggcccccagctaggccggctgttccacgcccagcaggacagcacaggggaggccctggtgaacttcactcaggcagaggtctacgctgggaatattctgtatgagcatgagatgccccccgagcccttttgggaggcccatgataccctagagctccagctgtcctcgccgcctgcccgggacgtggccgccacccttgctgtggctgtgtcttttgaggctgcctgtccccagcgccccagccacctctggaagaacaaaggtctctgggtccccgagggccagcgggccaggatcaccgtggctgctctggatgcctccaatctcttggccagcgttccatcaccccagcgctcagagcatgatgtgctcttccaggtcacacagttccccagccggggccagctgttggtgtccgaggagcccctccatgctgggcagccccacttcctgcagtcccagctggctgcagggcagctagtgtatgcccacggcggtgggggcacccagcaggatggcttccactttcgtgcccacctccaggggccagcaggggcctccgtggctggaccccaaacctcagaggcctttgccatcacggtgagggatgtaaatgagcggccccctcagccacaggcctctgtcccactccggctcacccgaggctctcgtgcccccatctcccgggcccagctgagtgtggtggacccagactcagctcctggggagattgagtacgaggtccagcgggcaccccacaacggcttcctcagcctggtgggtggtggcctggggcccgtgacccgcttcacgcaagccgatgtggattcagggcggctggccttcgtggccaacgggagcagcgtggcaggcatcttccagctgagcatgtctgatggggccagcccacccctgcccatgtccctggctgtggacatcctaccatccgccatcgaggtgcagctgcgggcacccctggaggtgccccaagctttggggcgctcctcactgagccagcagcagctccgggtggtttcagatcgggaggagccagaggcagcataccgcctcatccagggaccccagtatgggcatctcctggtgggcgggcggcccacctcggccttcagccaattccagatagaccagggcgaggtggtctttgccttcaccaacttctcctcctctcatgaccacttcagagtcctggcactggctaggggtgtcaatgcatcagccgtagtgaacgtcactgtgagggctctgctgcatgtgtgggcaggtgggccatggccccagggtgccaccctgcgcctggaccccaccgtcctagatgctggcgagctggccaaccgcacaggcagtgtgccgcgcttccgcctcctggagggaccccggcatggccgcgtggtccgcgtgccccgagccaggacggagcccgggggcagccagctggtggagcagttcactcagcaggaccttgaggacgggaggctggggctggaggtgggcaggccagaggggagggcccccggccccgcaggtgacagtctcactctggagctgtgggcacagggcgtcccgcctgctgtggcctccctggactttgccactgagccttacaatgctgcccggccctacagcgtggccctgctcagtgtccccgaggccgcccggacggaagcagggaagccagagagcagcacccccacaggcgagccaggccccatggcatccagccctgagcccgctgtggccaagggaggcttcctgagcttccttgaggccaacatgttcagcgtcatcatccccatgtgcctggtacttctgctcctggcgctcatcctgcccctgctcttctacctccgaaaacgcaacaagacgggcaagcatgacgtccaggtcctgactgccaagccccgcaacggcctggctggtgacaccgagacctttcgcaaggtggagccaggccaggccatcccgctcacagctgtgcctggccaggggccccctccaggaggccagcctgacccagagctgctgcagttctgccggacacccaaccctgcccttaagaatggccagtactgggtgtgaggcctggcctgggcccagatgctgatcgggccagggacaggcttgcccatgtcccgggccccattgcttccatgcctggtgctgtctgagtatccccagagcaagagagacctggagacaccaggggtggagggtcctgggagatagtcccaggggtccgggacagagtggagtcaagagctggaacctccctcagctcactccgagcctggagaactgcaggggccaaggtggaggcaggcttaagttcagtcctcctgccctggagctggtttgggctgtcaaaaccagggtaacctcctacatgggtcatgactctgggtcctgggtctgtgaccttgggtaagtcgcgcctgacccaggctgctaagagggcaaggagaaggaagtaccctggggagggaagggacagaggaagctattcctggcttttccactccaacccaggccaccctttgtctctgccccagagttgagaaaaaaacttcctcccctggttttttagggagatggtatcccctggagtagagggcaagaggagagagcgcctccagtctagaaggcataagccaataggataatatattcagggtgcagggtgggtaggttgctctggggatgggtttatttaagggagattgcaaggaagctatttaacatggtgctgagctagccaggactgatggagcccctgggggtgtgggatggaggagggtctgcagccagttcattcccagggccccatcttgatgggccaagggctaaacatgcatgtgtcagtggctttggagcaggttaggctggggctcatcgagggtctcaggccgaggccactgcggtgccagtgcccccctgaggactagggcaggcagctgggggcacttggttccatggagcctggataaacagtgctttggaggctctggacagctgtgtggtgtttgtgtcttaactatgcactgggcccttgtctgcgtcggcttgcatacagagggcccctggggtcggccctccggcctggcctcagccagtgggatggacagggccaggcaggcctctgaacttccacctcctggggcctcccagacctcctgtgcccccacctgtgtgggcaggtgggccagtcttcgggtgatgggaccaaaccccttcagttcagtagagaaaggctaggtcctctacaaagagctgcaagacaaaaattaaaataaatgctccccaccctagaaaaaaaaaaaaaaaa

SEQ ID NO: 21 - DNA, 5968 bp, Homo sapiens
gggataatgctcccgagaaggattctggcagcagttctcaaaggctagacttgagtggtattgctgcatatgcgctgattcttcagcttgtctctaaccgaggaagcattgattgggagctactcattcagaaaattaaaagaaagaagccagaaaatattatcaaccctttgagaacacgacacaacgaactttatattttaccacttccttgaatagttgcaggagaaataacaaggcattgaagaatggcagatgaacggaaagacgaagcaaaggcacctcactggacctcagcaccgctaacagaggcatctgcacactcacatccacctgagattaaggatcaaggcggacgaggggaaggacttgtccgaagcgccaatggattcccatacagggaggatgaagagggtgcctttggagagcatgggtcacagggcacctattcaaataccaaagagaatgggatcaacggagagctgacctcagctgacagagaaacagcagaggaggtgtctgcaaggatagttcaagtagtcactgctgaggctgtagcagtcctgaaagctgaacaagagaaagaagctcaacataaagaccagactgcagctctgcctttagcagctgaagaaacagctaatctgcctccttctccacccccatcacctgcctcagaacagactgtcacagtggaggaagatttacttacagcctcgaagatggagttccacgatcaacaggaattgactccctctacagctgagccttcagaccagaaggaaaaggagtcagagaagcaaagtagtcctggtgaagaccttaaacatgctgccttagtttctcagccagagacaactaaaacttaccctgataaaaaggacatgcaaggcacagaagaagaaaaagcacccctagctttgtttgggcacactcttgttgccagcctggaagacatgaaacagaagacagaaccaagccttgtagtacctggcattgacctccctaaagagcctccaactccaaaagaacaaaaggactggttcatcgaaatgccaacggaagcaaaaaaggatgagtggggtttagttgcccccatatctcctggccctctgactcccatgagggaaaaagatgtatttgatgatatcccaaaatgggaagggaaacagtttgattctcccatgccaagtccctttcaagggggaagcttcactcttcctttagatgtcatgaagaatgaaatagttacagaaacatcgccctttgcccctgcctttttacagccagatgacaaaaaatctctgcaacaaaccagtggcccagctactgccaaagatagttttaaaattgaagagccccatgaggctaaacctgacaaaatggcagaagcaccaccctcagaggcaatgaccttacccaaagatgctcacattccagttgtagaagaacatgttatggggaaagttttagaggaagaaaaggaggccataaatcaagagactgtgcagcaaagggatactttcacccccagtggacaggaacctatacttactgaaaaggaaactgagctgaagcttgaagaaaaaaccaccatttctgacaaagaagctgtgccaaaagagagtaaacccccaaaacctgcagatgaagaaataggcataattcagacctccacagagcacactttctcagaacagaaagaccaagagcctaccacagatatgttgaaacaggactcgttccctgtaagtttggagcaagcagttacagattcagccatgacctctaaaacactggagaaagccatgaccgaaccatctgcattaattgaaaagagctcaattcaggaactttttgaaatgagagttgatgacaaagataagattgaaggagttggagctgcaacatcagctgagcttgatatgccattttatgaagataaatcaggaatgtccaagtactttgaaacatctgccttgaaagaagaagcaacaaaaagcattgagccaggcagtgattactatgaactgagtgacactagagaaagtgtccatgagtctattgataccatgtctcccatgcataaaaatggtgacaaggagtttcaaacaggaaaagaatcccagcccagtcctccagcacaagaagcagggtacagcactctcgcacagagttatccatcagatttacctgaagaacccagttctcctcaagaaagaatgttcactattgatccaaaagtgtatggagagaaaagggacctccacagtaagaataaggatgatttgacccttagcaggagtttaggacttggtggtaggtctgcaatagaacaaagaagcatgtcaatcaatttgccgatgtcttgcctagattccatagcccttggatttaactttggtcggggacatgatctttctcctctggcttccgatattctaaccaacactagtggaagtatggatgaaggggatgattaccttccagccaccacacctgcactggagaaagccccttgcttccctgtagaaagcaaagaggaagaacagatagagaaagtaaaagctactggagaagaaagtactcaagcggagatatcatgtgagtctcctttcctagccaaagatttttacaaaaatggtactgtcatggcacctgaccttcctgaaatgctagatctggcaggcacaaggtcaagattggcttctgtgagtgcagatgctgaggttgccaggaggaaatcagtcccatcagagactgtggttgaggatagtcgtactggcttgcccccggtaactgatgaaaaccatgtcattgtaaaaacggacagtcagctcgaagacctgggctactgtgtgttcaataagtacacagtcccattgccatcacctgttcaagacagtgagaatttatcaggggagagtggtaccttttacgaaggcactgatgataaagttcgaagagatttggccacagacctttcactgattgaagtgaaactggcagcagccggaagagtcaaagatgagttcagtgttgacaaagaagcatccgcgcatatctctggtgacaaatcaggactgagtaaggagtttgaccaagagaagaaagctaatgataggttggatactgtactagaaaagagtgaagaacatgctgattcaaaagaacatgccaagaaaactgaagaggctggtgatgaaatagaaacattcggattaggagtaacctatgagcaagctttggccaaagatttgtcaataccaacagatgcatcctctgagaaagcagagaagggtcttagttcagttccagagatagctgaggtagaaccatccaaaaaggtggaacaaggtctggattttgctgtccagggtcaactagatgttaaaattagtgactttggacagatggcttcagggctaaacatagatgatagaagggcaacagagctaaaacttgaggctacacaggacatgaccccctcatccaaagcaccgcaggaggcagatgcatttatgggtgttgagtctggccacatgaaagaaggcactaaagttagtgagacagaagtcaaacagaaggtggccaagcctgacttggtgcaccaggaggctgtagacaaggaggagtcctatgaatctagtggtgagcatgaaagtctcaccatggagtccttgaaagctgatgagggcaagaaggaaacatctccagaatcatctctaattcaagatgagattgccgtcaaattgtcagtggaaataccttgcccacctgctgtttcagaggctgatttagccacagatgagagagctgatgtccagatggaatttattcaggggccaaaagaagaaagcaaagagaccccagatatatccatcacgccttctgatgttgcagagccattgcatgaaacgatcgtatctgaaccagcagagattcagagtgaggaagaagagatagaagcccagggagaatatgataaactgctcttccgctcagacacccttcagataactgacctgggtgtctcaggtgccagggaggaatttgtggagacctgcccaagtgaacacaaaggagtgattgagtctgttgtgaccatcgaggatgatttcatcactgtagtgcaaaccacaactgatgaaggggagtcagggtcccacagcgtgcgttttgcagccctagagcagcctgaggtggaaaggagaccatctcctcatgatgaagaagagtttgaagtagaagaggcagctgaagcccaggcagaacccaaagatggttccccagaggctccagcttcccctgagagagaagaggttgcactttctgaatataagacagaaacctatgacgattacaaagatgagaccaccattgacgactccatcatggacgctgacagcctctgggtggacactcaagatgatgataggagcatcatgacagaacagttagaaactattcctaaagaggagaaagctgaaaaggaagctcggagatcatctcttgagaaacatagaaaagaaaagccttttaaaaccgggagaggcagaatttccactcctgaaagaaaagtagctaaaaaggaacctagcacagtctccagagatgaagtgagaaggaaaaaagcagtttataagaaggctgaacttgctaaaaaaacagaagttcaggcccactctccctccaggaaattcattttaaaacctgctatcaaatatactagaccaactcatctctcctgtgttaagcggaaaaccacagcagcaggtggggaatcagctctggctcccagtgtatttaaacaggcaaaggacaaagtctctgacggagtaaccaagagcccagaaaagcgctcttctctcccaagaccttcctccattctccctcctcggcgaggtgtgtcaggagacagagatgagaattccttctctctcaacagttctatctcttcttcagcacggcggaccaccaggtcagagccaattcgcagagcaggaaagagtggtacctcaacacccactacccctgggtctactgccatcactcctggcaccccaccaagttattcttcacgcacaccaggcactcctggaacccctagctatcccaggacccctcacacaccaggaacccccaagtctgccatcttggtgccgagtgagaagaaggtcgccatcatacgtactcctccaaaatctcctgcgactcccaagcagcttcggcttattaaccaaccactgccagacctgaagaatgtcaaatccaaaatcggatcaacagacaacatcaaataccagcctaaaggggggcaggtacaaattgttaccaagaaaatagacctaagccatgtgacatccaaatgtggctctctgaagaacatcgacaggccaggtggcggacgtgtgaaaattgagagtgtaaaactagatttcaaggaaaaggtccaagctaaagttggttctcttgataatgctcatcatgtacctggaggtggtaatgtcaagattgacagccaaaagttgaacttcagagagcatgctaaagcccgtgtggaccatggggctgagatcattacacagtccccaggcagatccagcgtggcatcaccccgacgactcagcaatgtctcctcgtctggaagcatcaacctgctcgaatctcctcagcttgccactttggctgaggatgtcactgctgcactcgctaagcagggcttgtgaatatttctcatttagcattgaaataataatatttaggcatgagctcttggcaggagtgggctctgagcagttgttatattcattctttataaaccataaataaataatctcatccccaaactgtagtaattgttacaattttctatttaaaaaatgaatagtacatgcagaaattgacctgatttccatttgcaacaggaagacactggctttactgggttcaattggacaattatttt

SEQ ID NO: 22 - DNA, 1488 bp, Homo sapiens
gcaggatggagaggagacgcatcacctccgctgctcgccgctcctacgtctcctcaggggagatgatggtggggggcctggctcctggccgccgtctgggtcctggcacccgcctctccctggctcgaatgccccctccactcccgacccgagtggatttctccctggctggggcactcaatgctggcttcaaggagacccgggccagtgagcgggcagagatgatggagctcaatgaccgctttgccagctacatcgagaaggttcgcttcctggaacagcaaaacaaggcgctggctgctgagctgaaccagctgcgggccaaggagcccaccaagctggcagacgtctaccaggctgagctgcgagagctgcggctgcggctcgatcaactcaccgccaacagcgcccggctggaggttgagagggacaatctggcacaggacctggccactgtgaggcagaagctccaggatggaaccaacctgaggctggaagccgagaacaacctggctgcctatagacaggaagcagatgaagccaccctggcccgtctggatctggagaggaagattgagtcgctggaggaggagatccggttcttgaggaagatccacgaggaggaggttcgggaactccaggagcagctggcccgacagcaggtccatgtggagcttgacgtggccaagccagacctcaccgcagccctgaaagagatccgcacgcagtatgaggcaatggcgtccagcaacatgcatgaagccgaagagtggtaccgctccaagtttgcagacctgacagacgctgctgcccgcaacgcggagctgctccgccaggccaagcacgaagccaacgactaccggcgccagttgcagtccttgacctgcgacctggagtctctgcgcggcacgaacgagtccctggagaggcagatgcgcgagcaggaggagcggcacgtgcgggaggcggccagttatcaggaggcgctggcgcggctggaggaagaggggcagagcctcaaggacgagatggcccgccacttgcaggagtaccaggacctgctcaatgtcaagctggccctggacatcgagatcgccacctacaggaagctgctagagggcgaggagaaccggatcaccattcccgtgcagaccttctccaacctgcagattcgagaaaccagcctggacaccaagtctgtgtcagaaggccacctcaagaggaacatcgtggtgaagaccgtggagatgcgggatggagaggtcattaaggagtccaagcaggagcacaaggatgtgatgtgaggcaggacccacctggtggcctctgccccgtctcatgaggggcccgagcagaagcaggatagttgctccgcctctgctggcacatttccccagacctgagctccccaccaccccagctgctcccctccctcctctgtccctaggtcagcttgctgccctaggctccgtcagtatcaggcctgcc

(19) United States
(12) Patent Application Publication
(10) Pub. No.: US 2009/0304647 A1
(43) Pub. Date: Dec. 10, 2009

(54) Production of Neural Stem Cells from Bone Marrow Tissue and Use Thereof

(75) Inventors: Tingyu Qu, Chicago, IL (US); Ke Ma, Chicago, IL (US); Guangbin Shi, Hinsdale, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Board of Trustees of the University of Illinois, Urbana, IL (US)

(21) Appl. No.: 12/476,029

(22) Filed: Jun. 1, 2009

Related U.S. Application Data
(60) Provisional application No. 61/057,695, filed on May 30, 2008.
(60) Provisional application No. 61/061,374, filed on Jun. 13, 2008.

(51) Int. Cl. A61K 45/00; C12N 5/06; C12N 5/08; C12N 5/02; A61P 25/28

(52) U.S. Cl. 424/93.7; 435/377; 435/372; 435/406

(57) ABSTRACT

The invention provides reagents and methods for preparing mammalian mesenchymal-derived neural stem cells, especially autologous mesenchymal-derived neural stem cells, compositions thereof, and methods for using and administering the cells in a patient in need thereof.

This application relates to and claims the benefit of priority to U.S. Provisional Application Ser. Nos. 61/057,695, filed May 30, 2008 and 61/061,374, filed Jun. 13, 2008, the disclosures of both of which are incorporated herein by reference in their entireties.

### BACKGROUND OF THE INVENTION

This invention relates to stem cells, particularly neural stem cells and specifically human neural stem cells. Stem cells are often defined as self-renewing and multipotent, with the ability to generate diverse types of differentiated cells. For at least these reasons, neural stem cells (NSCs) show promise for treating neurological deficits, or any loss or diminishment of neural tissue function due to age, disease, trauma or other factors. Thus, one important focus of stem cell replacement therapies has been neurological disorders, and accordingly neural stem cells, particularly fetal neural stem cells, have been a major research target.

During central nervous system (CNS) development, multipotent neural stem cells (MNSCs), also known as neural stem cells (NSCs), proliferate and give rise to transiently-dividing progenitor cells that eventually differentiate into the cell types that compose the adult brain, including neurons, astrocytes and oligodendrocytes. NSCs have been isolated from several mammalian species, including mice, rats, pigs and humans. See, e.g., International Application, Publication Nos. WO 93/01275, WO 94/09119, WO 94/10292, WO 94/16718 and Cattaneo et al., 1996, Mol. Brain. Res. 42: 161-66. NSCs from embryonic and adult rodent CNS have been isolated and further propagated in vitro in a variety of culture systems. See, e.g., Frolichsthal-Schoeller et al., 1999, NeuroReport 10: 345-351; Doetsch et al., 1999, Cell 97: 703-716. Human fetal brain-derived NSCs have been cultured using serum-free medium supplemented with epidermal growth factor (EGF) and/or basic fibroblast growth factor (bFGF; see, e.g., Svendsen et al., 1998, J. Neuroscience Methods 85: 141-152; Carpenter et al., 1999, Experimental Neurology 158: 265-278). NSCs cultured utilizing serum-free, mitogen-supplemented culturing methods generally form substantially undifferentiated, clustered aggregates. Upon removal of the mitogen(s) and provision of a substrate, the stem cells differentiate into neurons, astrocytes and oligodendrocytes.

While the synaptic connections involved in neural circuits are continuously altered throughout the life of the individual (due to synaptic plasticity and cell death), neurogenesis, the generation of new neurons, was thought to be complete early in the postnatal period. The discovery of NSCs in the adult brain (see, e.g., Alvarez-Buylla et al., 1997, J. Neurobiology 33: 585-601; Gould et al., 1999, Science 286: 548-552) has brought significant changes in theories of neurogenesis, as the presence of NSCs in the adult brain suggests that regeneration of neurons can occur throughout life. Nevertheless, age, physical and biological trauma or neurodegenerative disease-associated loss of brain function can far outweigh any potential restorative effects due to endogenous neurogenesis. Accordingly, transplantation of NSCs is a potentially valuable treatment for those suffering from the loss of, or loss of appropriate, brain function due to age, physical and biological trauma or neurodegenerative disease.

Due to the advancing average age of the population, and concomitantly increased incidence of neurological deficit that accompanies advancing age, treatment of neurodegenerative diseases has become a major concern. Such diseases, including Alzheimer's disease, Huntington's chorea and Parkinson's disease, have been linked to neuronal degeneration at specific locations in the brain, related to an inability of the brain region to synthesize and release neurotransmitters that are vital to neuronal signaling.

The objective of most CNS therapies is to regain the particular chemical function or enzymatic activity lost due to cellular degeneration. Administration of pharmaceutical compositions has been the main treatment for CNS dysfunction; however, this type of treatment is not optimal, due to the existence of complications and limitations in the capacity for drugs to be transported across the blood-brain barrier, and drug-tolerance acquired by patients to whom these drugs are administered for long periods.

Transplanting multipotent neural stem cells may avert the need not only for constant drug administration, but also for complicated drug delivery systems necessitated by the blood-brain barrier. In practice, however, significant limitations exist in this technique as well. First, cells used for transplantation that carry cell surface molecules of a differentiated cell from a donor can induce an immune reaction in the recipient, a problem that is exacerbated by the physical damage caused by injection of cells directly into the affected area of the brain. In addition, the neural stem cells must be at a developmental stage where they are able to form neural connections with neighboring cells. For these reasons, initial studies on neurotransplantation centered on the use of fetal cells.

Mammalian fetal brain tissue has proven to possess higher survival capability partly due to its reduced susceptibility to anoxia compared to adult neurons. An additional factor favoring survival of fetal cells is the lack of cell surface markers on fetal cells, whose presence may lead to rejection of grafted tissue from recipients. However, although the brain is considered an immunologically privileged site, some rejection of even fetal tissue can still occur.

Additionally, the use of large quantities of aborted fetal tissue presents other difficulties as well. Fetal CNS tissue is composed of more than one cell type, and thus is not a well-defined tissue source. Further, it is difficult to maintain an adequate and constant supply of fetal tissue for transplantation. For example, in the treatment of MPTP (1-methyl-4-phenyl-1,2,3,6-tetrahydropyridine)-induced Parkinsonism, tissue from as many as 6 to 8 fetuses can be required for successful implantation into the brain of a single patient. Another problem is the potential for contamination during fetal tissue preparation. Expensive diagnostic testing for bacterial or viral infection is required for each fetus used. But even comprehensive diagnostic testing might not uncover all infected tissue. For example, negative immunoreactivity to HIV antigens does not guarantee that the fetal tissue is HIV-free, because antibodies to the virus are generally not present until several weeks after infection. The use of large quantities of aborted fetal tissue also presents controversial ethical issues in addition to the risk of immune rejection, risk of infection, the heterogeniety of the fetal tissue, and the short supply of fetal tissue as mentioned above.

While currently available transplantation approaches represent an improvement over other available treatments for neurological disorders, they suffer from significant drawbacks. These include an inability of the transplant to fully integrate into the host tissue, and the unavailability of suitable cells in unlimited amounts from a reliable source for grafting. Studies utilizing intra-tissue injection of dissociated and partially differentiated NSCs have shown little success (see, e.g., Benninger et al., 2000, Brain Pathol. 10: 330-341; Blakemore et al. 2000, Cell Transplant 9: 289-294; Rosser et al., 2000, Eur. J. Neurosci. 12: 2405-2413; Rubio et al., 2000, Mol. Cell. Neurosci. 16: 1-13), partly because dissociation of clusters of NSCs induces immediate senescence of NSCs. See, e.g., Svendsen et al., 1998, J. Neurosci. Methods 85: 141-152. Further, in addition to adverse immune responses due to foreign tissue being introduced into the brain, trauma caused by the physical introduction of cells directly into the damaged area can induce the recruitment of immune cells by the host that can eliminate the transplanted cells. Thus, significant problems with the use of NSCs to ameliorate neurological deficits remain.

Therefore, there exists a need in the art for improved neural stem cells and methods for introducing the neural stem cells to diseased, aged or damaged mammalian brain. Specifically, there exists a need for improved methods for introducing neural stem cells that do not elicit adverse immune response to a mammal in need thereof. There also remains a need in the art for a reliable source of unlimited numbers of cells for transplantation, particularly cells that are specifically adapted for and capable of proliferation, migration, and differentiation in mammalian brain or other tissues when introduced thereto. Furthermore, there exists a need for a safe, feasible, and effective neuroreplacement therapy for treating neural deficits or disorders.

### SUMMARY OF THE INVENTION

The invention provides methods and reagents for making and using mesenchymal-derived neural stem cells, either heterologous or autologous mesenchymal-derived neural stem cells, for transplantation to a mammal, preferably a human and particularly a human patient in need thereof. Specifically, the invention provides methods and reagents for making and administering autologous mesenchymal-derived neural stem cells. In particular, these methods and reagents are capable of reversing the effects of neural degeneration due to aging, trauma, or other underlying diseases without the drawbacks (including inefficient drug delivery or undesirable immune responses) of currently available therapies.

In one aspect, the invention provides methods for making mesenchymal-derived neural stem cells from a mammal's bone marrow, the method comprising the steps of isolating mesenchymal stem cells from bone marrow and culturing the mesenchymal stem cells in a cell-free human neural stem cell conditioned medium to produce mesenchymal-derived neural stem cells, wherein the mesenchymal-derived neural stem cells are capable of differentiating into neural cells. In certain embodiments, the mammal is a human. In certain other embodiments, the human has a neurological disease, deficit, defect, deficiency or disorder. In certain embodiments, the neurological disease, deficit, defect, deficiency or disorder includes without limitation traumatic brain injury, spinal cord injury, Parkinson's disease, Alzheimer's disease, amyotrophic lateral sclerosis, motor neuron diseases, stroke or sequelae of stroke. Accordingly, certain embodiments of this aspect of the invention provide methods for making autologous mesenchymal-derived neural stem cells. In certain other embodiments, the bone marrow is fresh or frozen bone marrow.

In accordance with this aspect of the invention, the cell-free human neural stem cell conditioned medium is obtained by a process comprising the steps of culturing human neural stem cells in serum-free medium containing B27 or N2 in the presence of bFGF (basic fibroblast growth factor) and EGF (epidermal growth factor) and optionally LIF (leukemia inhibitory factor), and collecting the cell-free conditioned medium after at least 2 days, at least 3 days, especially after 2-3 days in culture. In particular embodiments, the human stem cells are maintained in culture for 50-60 days and the cell-free conditioned medium is collected every 2-3 days. In certain embodiments, the serum-free medium contains B27.

In certain particular embodiments, the mesenchymal stem cells are cultured in the cell-free human neural stem cell conditioned medium in the presence of at least one mitogenic growth factor, including without limitation, bFGF, EGF, LIF, PDGF (platelet-derived growth factor) or FGF8 (fibroblast growth factor 8). In other particular embodiments, the mitogenic growth factor is bFGF or EGF. In certain particular embodiments, the mesenchymal stem cells are cultured in the cell-free human neural stem cell conditioned medium in the absence of BrdU. In advantageous embodiments, the mesenchymal stem cells cultured in the cell-free human neural stem cell conditioned medium described herein become neural stem cells committed to neural lineage differentiation.

In another aspect, the invention provides isolated mesenchymal-derived neural stem cells produced by the method described herein comprising the steps of isolating mesenchymal stem cells from bone marrow of a mammal and culturing the mesenchymal stem cells in a cell-free human neural stem cell conditioned medium to produce mesenchymal-derived neural stem cells, wherein the mesenchymal-derived neural stem cells are capable of differentiating into neural cells. In certain embodiments, the mammal is a human, especially a human who has a neurological disease, deficit, defect, deficiency or disorder. In certain embodiments, the neurological disease, deficit, defect, deficiency or disorder is traumatic brain injury, spinal cord injury, Parkinson's disease, Alzheimer's disease, amyotrophic lateral sclerosis, motor neuron diseases, stroke or sequelae of stroke. In certain other embodiments, the bone marrow is fresh or frozen bone marrow.

The mesenchymal-derived neural stem cells of this aspect of the invention are capable of forming a neurosphere. In certain particular embodiments, the mesenchymal-derived neural stem cells express early or embryonic neural marker stage-specific embryonic antigen 4 (SSEA4), BF1, nuclear receptor related protein 1 (Nurr1), Neurogenic Differentiation (NeuroD), POU domain transcription factor 4 (OCT4), Nanog, human paired box gene 6 (Pax6), Sex determining region Y-box 1 (Sox1), or Nestin. In further particular embodiments, the mesenchymal-derived neural stem cells do not exhibit action membrane potential of mature neurons in response to current pulses.

The mesenchymal-derived neural stem cells of the invention are committed to the neural lineage. In accordance with this aspect of the invention, the mesenchymal-derived neural stem cells are capable of differentiating into neural cells in a basal medium. In certain embodiments, the basal medium is supplemented with B27 or N2. In certain particular embodiments, the basal medium is supplemented with B27. In certain other particular embodiments, the basal medium further comprises at least one neurotrophic factor such as neural growth factor (NGF), brain-derived neurotrophic factor (BDNF), or glial cell line-derived neurotrophic factor (GDNF). In other particular embodiments, the mesenchymal-derived neural stem cells are capable of differentiating into neural cells when administered into a patent's central nervous system (CNS). In further embodiments, the mesenchymal-derived neural stem cells are capable of differentiating into neural cells that express glial fibrillary acidic protein (GFAP), NG2, beta III-tubulin, microtubule-associated protein 2 (MAP2), or neuron nuclear antigen (NeuN), or are immunopositive for GalC, GABA or glutamate.

In a further aspect, the invention provides pharmaceutical compositions or cellular preparations comprising mesenchymal-derived neural stem cells described herein. In certain embodiments, the mesenchymal-derived neural stem cells are derived from the bone marrow of a human, particularly a human having a neurological disease, deficit, defect, deficiency or disorder. In additional embodiments, pharmaceutical compositions of this invention further comprise at least one pharmaceutically-acceptable carrier, excipient or diluent.

In a further aspect, the invention provides methods for treating a patient having a neurological disease, deficit, defect, deficiency or disorder, the method comprising administering a therapeutically effective amount of the mesenchymal-derived neural stem cells of the invention to the patient, wherein the mesenchymal-derived neural stem cells differentiate to neural cells in a tissue-specific manner that reduces the neurological disease, deficit, defect, deficiency or disorder in the patient. In certain embodiments, the patient is a human. In certain particular embodiments, the mesenchymal-derived neural stem cells are autologous mesenchymal-derived neural stem cells derived from mesenchymal stem cells isolated from the patient's bone marrow.

In accordance with this aspect of the invention, in certain embodiments, the neurological disease, deficit, defect, deficiency or disorder includes without limitation traumatic brain injury, spinal cord injury, Parkinson's disease, Alzheimer's disease, amyotrophic lateral sclerosis, motor neuron diseases, stroke or sequelae of stroke. In certain other embodiments, the patient suffers from tissue damage in the neural tissue. In other embodiments, the mesenchymal-derived neural stem cells used in the methods of treatment according to the invention migrate to the area of tissue damage, differentiate in a tissue-specific manner at the area of tissue damage, and function in a manner that ameliorate the neurological disease, deficit, defect, deficiency or disorder.

In another aspect, the invention provides methods for treating a patient having a neurological disease, deficit, defect, deficiency or disorder, comprising administering to the patient in need of such treatment a pharmaceutical composition or a cellular preparation of a therapeutically effective amount of mesenchymal-derived neural stem cells. In certain embodiments, the patient is a human. In certain particular embodiments, the mesenchymal-derived neural stem cells are autologous mesenchymal-derived neural stem cells derived from mesenchymal stem cells isolated from the patient's bone marrow.

In yet another aspect, the invention provides mesenchymal-derived neural stem cells for use in therapy in treating a patient having a neurological disease, deficit, defect, deficiency or disorder. In a related aspect, the invention provides methods for using mesenchymal-derived neural stem cells to prepare a pharmaceutical composition or formulation for treating a neurological disease, deficit, defect, deficiency or disorder. In particular embodiments, the mesenchymal-derived neural stem cells are derived from the bone marrow of the patient. In further embodiments, the patient is a human.

In a further aspect, the invention provides methods for differentiating the mesenchymal-derived neural stem cells to neural cells comprising the step of culturing the mesenchymal-derived neural stem cells of this invention in a basal medium to allow neural differentiation of the mesenchymal-derived neural stem cells to neural cells. In certain particular embodiments, the basal medium does not contain any growth factors or neurotrophic factors. In certain other particular embodiments, the basal medium is further supplemented with B27 or N2. In other embodiments, the basal medium further comprises at least one neurotrophic factor neural growth factor (NGF), brain-derived neurotrophic factor (BDNF), or glial cell line-derived neurotrophic factor (GDNF). In another advantageous aspect, the invention provides methods for differentiating the mesenchymal-derived neural stem cells to neural cells comprising the step of administering the mesenchymal-derived neural stem cells of the invention into a patient's central nervous system.

In yet another aspect, the invention provides a cell-free human neural stem cell conditioned medium, wherein said conditioned medium is obtained by a process comprising the steps of culturing human neural stem cells in a serum-free medium containing B27 or N2 in the presence of bFGF and EGF and optionally LIF, and collecting the cell-free conditioned medium after at least 2 days, at least 3 days, or after 2-3 days in culture. In particular embodiments, the human neural stem cells are cultured for no more than 50-60 days in culture, and the conditioned medium is collected every 2-3 days. In a further aspect, the invention provides methods of preparing a cell-free human neural stem cell conditioned medium, said method comprising steps of culturing human neural stem cells in a serum-free medium containing B27 or N2 in the presence of bFGF and EGF, and collecting the cell-free conditioned medium after at least 2 days, at least 3 days, or after 2-3 days in culture. In certain particular aspects, the human neural stem cells are cultured in the serum-free medium further comprising LIF.

In yet another aspect, the invention provides kits for making mesenchymal-derived neural stem cells, said kits comprising a cell-free human neural stem cell conditioned medium of the invention and instructions for use thereof. In certain particular embodiments, the kit further comprises at least one mitogenic factor. In other particular embodiments, the mitogenic growth factor is bFGF, EGF, LIF, PDGF or FGF8.

Specific embodiments of the invention will become evident from the following more detailed description of certain preferred embodiments and the claims.

### BRIEF DESCRIPTION OF THE DRAWINGS

FIG. 1 shows a photomicrograph of fluorescent immunostaining of cells and images of ethidium bromide staining of electrophoretical gel analysis indicating that bone marrow mesenchymal-derived neural stem cells express markers for early embryonic cells: Pax6; OCT 4; and DAPI.

FIG. 2A-2D show photomicrographs of fluorescent immunostaining of cells indicating neural differentiation of bone marrow mesenchymal-derived neural stem cells cultured in a basal culture medium supplemented with B27. The bone marrow mesenchymal-derived neural stem cells differentiated into neural cells and glial cells in a basal culture medium supplemented with B27, and expressed an early neuron marker (beta III-tubulin) and a glial cell and astrocyte marker (glial fibrillary acidic protein (GFAP)) (FIG. 2A); an oligodendrocyte progenitor cell marker (NG2, top panel of FIG. 2B) and an oligodendrocyte marker (Gal-C, bottom panel of FIG. 2B); neuron markers (gamma-aminobutyric acid (GABA), top panel of FIG. 2C; and glutamate, bottom panel of FIG. 2C); and a mature neuron marker (MAP2, FIG. 2D). The nuclei of the cells are identified by counter-staining with the nucleus marker 4′,6-diamidino-2-phenylindole (DAPI).

FIG. 3 shows a representative graph illustrating the effects of glutamate and KCl on Ca²⁺ tracing as a function of time in the mesenchymal-derived neural stem cells at day six after differentiation in the basal culture medium supplemented with B27.

FIGS. 4A-4D depict graphs demonstrating neuron-like membrane properties of cells differentiated in vitro from human mesenchymal-derived NSCs (FIGS. 4A-4B) as compared to non-differentiated mesenchymal-derived NSC (FIG. 4C). FIGS. 4A-4C: patch-clamp recordings. FIG. 4D: plot of the steady-state current-voltage relationship for the cells in FIGS. 4A-4C.

FIG. 5 shows microphotographs of immunofluorescent staining of astrocyte marker GFAP (FIG. 5A), and neuronal markers 03111-tubulin (FIG. 5B) and NeuN (FIG. 5C) in normal mouse brain 4 weeks after transplantation of human bone marrow mesenchymal-derived NSCs.

### DETAILED DESCRIPTION OF THE INVENTION

This invention provides methods and reagents for making and using mesenchymal-derived neural stem cells that can differentiate into neural cells in a tissue-specific manner, particularly autologous mesenchymal-derived neural stem cells that are derived from a mammal's bone marrow and can be administered to the mammal in need of such cell transplantation. Compositions of such autologous, mesenchymally-derived neural stem cells, including pharmaceutical compositions, are also provided.

NSCs have been isolated from embryonic and fetal mammalian and human brains and propagated in vitro in a variety of culture systems (Doetsch et al., 1999, Subventricular zone astrocytes are neural stem cells in the adult mammalian brain. Cell 97:703-16, Johansson et al., 1999, Cell 96:25-34, Svendsen et al., 1998, J Neurosci Methods 85:141-52). A system for proliferating human neural stem cells (hNSCs) in serum-free culture medium containing human bFGF and human EGF also has been reported (Kim et al., 2002, Proc Natl Acad Sci USA 99: 4020-4025, Qu et al., 2001, NeuroReport 12: 1127-1132). Further, transplantation of hNSCs into experimental animals has been described (Qu et al., 2001, Id.; Qu et al., 2005, 35th Annual Meeting in Washington, D.C., November, 2005). All references cited throughout the application are herein incorporated by reference for any purposes.

However, challenges existed in the art of stem cell therapies using stem cells derived from embryonic/fetal tissue sources. Stem cell therapies using embryonic sources face challenges such as ethical issues, technical difficulties in cell isolation, and the need for long-term immunosuppressant administration to transplant recipients; the limitations of using fetal tissue sources have been set forth above. These challenges have hindered the applicability of hNSCs for human use.

Bone marrow (BM) contains stem cells involved not only in hematopoiesis but also for production of a variety of nonhematopoietic tissues. A subset of stromal cells in bone marrow, mesenchymal stem cells (MeSCs), is capable of self-renewing and producing multiple mesenchymal cell lineages, including bone, cartilage, fat tendons, and other connective tissues (Majumdar et al., 1998, J Cell Physiol. 176:57-66, Pereira et al., 1995, Proc Natl Acad Sci USA. 92: 4857-61, Pittenger et al., 1999, Science 284:143-7). Bone marrow mesenchymal stem cells normally are not committed to the neural lineage in differentiation. Although adult stem cells continue to possess some degrees of multipotency, cell types produced from adult stem cells are thought to be limited by their tissue-specific character. To overcome this barrier, it is necessary to alter the cell lineage of these adult stem cells.

Despite this general resistance for adult stem cells to cross such tissue-specific limitations, recently it has been shown that MeSCs can differentiate into a diverse family of cell types unrelated to their phenotypical embryonic origin, under certain experimentally-defined conditions. These cell types include muscle and heptocytes (Imasawa et al., 2001, J Am Soc Nephrol. 12:1401-9, Liechty et al., 2000, Nat. Med. 6(11):1282-6, Petersen et al., 1999, Science 284:1168-70, Makino et al., 1999, J Clin Invest. 103:697-705). Several groups of researchers have reported differentiation of MeSCs to neurons and glia in defined cell culture conditions (Woodbury et al, 2000, J Neurosci Res. 61:364-70, Sanchez-Ramos et al., 2000, Exp Neurol. 164:247-56). Some transplantation studies also showed neural and glia differentiation of MeSCs in the brain (Chen et al., 2000, Neuropharmacology 39:711-6, Kopen et al., 1999, Proc Natl Acad Sci USA. 96:10711-6, Chopp et al., 2000, Neuroreport 11:3001-5, Li et al., 2000, J Cereb Blood Flow Metab. 20:1311-9). In spite that great efforts have been made to produce neural cells from hMeSCs, the regulatory mechanisms of tissue-specific stem cell fate decisions remain unclear.

It was previously shown that BrdU pre-treated hMeSCs, when co-cultured with differentiating or differentiated hNSCs developed into neurons and glia (as set forth in co-owned U.S. patent application Ser. No. 10/345,126, published under Patent Application Publication No. 2003/0219898, which is incorporated herein by reference in its entirety); see also Qu et al., 2004, Restorative neurology and Neuroscience, 22: 459-468). In these prior studies, hNSCs differentiating in 12-well culture plates in a basal medium (Invitrogen, Carlsbad, Calif.) for five days contained mixed neural cell populations, including neural precursors, neurons, and astrocytes at different developmental stages. As disclosed previously, hMeSCs treated with 10 μM BrdU for 5 days were transferred onto a tissue culture membrane insert of 0.4 μM pore size (Falcon, BD Biosciences, San Jose, Calif.) and placed on top of differentiating or differentiated hNSCs in basal media. Immunocytochemical examination seven days post-coculture revealed that these BrdU-treated hMeSCs differentiated into beta 1′-tubulin and GFAP-immunopositive cells, something that was not observed to occur spontaneously (i.e., in the absence of co-culture). Moreover, without BrdU pretreatment, hMeSCs did not express either βIII-tubulin or GFAP even after co-culture with differentiating hNSCs. However, BrdU treatment alone was insufficient to produce neural cells. In this co-culture system, hMeSCs were cultured on the membrane insert, separating the hMeSCs from the hNSCs, thus the possibility of fusion between hMeSCs and hNSCs was excluded. BrdU-pretreated hMeSCs are capable of developing into neurons and astrocytes in vivo 4-6 weeks after transplantation into rat and mouse brains. (Qu et al., 2004, Id.) Thus, in these prior studies, both BrdU and co-culture were required for neural differentiation.

Without being bound by any particular theory for the mechanism, these results from prior studies are consistent with BrdU-mediated modulation of DNA methylation in hMeSCs. Such DNA methylation could initiate the observed neural lineage change when the cells were exposed to factors released from differentiating hNSCs. BrdU, an analog of thymidine, can integrate into chromosomal DNA by replacing thymidine and thereby modify the status of DNA methylation in those dividing hMeSCs, while differentiating hNSCs may produce cellular bio-factors that affect the fate decision of hMeSCs. (Call et al., 1991, Mutat Res. 248:101-14, Suzuki et al., 2001, Exp Cell Res. 266:53-63). Thus, in the culture system of these prior studies, both epigenetic modifications (BrdU treatment) and microenvironmental influences (co-culturing with differentiating NSCs) played a crucial role in the neural differentiation of hMeSCs.

However, the long term effects of the BrdU treatment on the cells and the long term effects of administering those BrdU-treated cells remain unknown. Advantageously, mesenchymal-derived neural stem cells of this invention were derived from hMeSCs cultured in a serum-free hNSC conditioned medium (hNSCcm) without BrdU treatment and without the addition of other undefined exogenous chemical factors, which is similar to the physiological conditions required for the production of clinical-grade cells. (Ma et al., 36^{th} Annual Meeting in Atlanta, Ga., October, 2006). The resulting mesenchymal-derived human neural stem cells produced according to the methods of this invention are thus safer and the invention provides more reliable methods for differentiating hMeSCs into neural cells.

It will be understood by one of ordinary skill in the art that BrdU can be used to label and distinguish injected mesenchymal-derived neural stem cells of this invention from host cells after transplantation. However, the methods of making mesenchymal-derived neural stem cells of this invention do not require the treatment of mesenchymal stem cells with BrdU. The concentration of BrdU and especially the time period of exposure to BrdU applied in the instant application for labeling purpose (1 μM BrdU for 48 hr) is less than those used in the prior studies described in U.S. 2003/0219898. The dose and time period of BrdU exposure in the instant application is insufficient to render the mesenchymal stem cells to become the mesenchymal-derived neural stem cells of this invention. In addition, in the instant application, BrdU is added to the mesenchymal-derived neural stem cells after the cells have committed to the neural lineage, i.e., after the mesenchymal stem cells have been first cultured in the hNSC conditioned medium. On the other hand, the method described in the prior studies requires addition of BrdU at higher concentration (e.g., 2 μM or 10 μM) for longer period of time (e.g., 3 or 5 days) to the mesenchymal stem cells, before the cells become capable of neural differentiation. Thus, the methods of making mesenchymal-derived neural stem cells of this invention do not require BrdU to alter the mesenchymal stem cells to neural stem cells committed to neural lineage differentiation. In further contrast to the prior studies, once committed to neural lineage differentiation, the mesenchymal-derived neural stem cells of this invention do not require co-culture with differentiating or differentiated neural stem cells of the CNS to differentiate into neural cells.

This invention provides methods for making neural stem cells from easily isolated and abundant stem cells, particularly mesenchymal stem cells from the bone marrow. The mesenchymal-derived neural stem cells of the invention are committed to neural lineage differentiation, capable of differentiation into neural-lineage cells including neurons, astrocytes, and oligodendrocytes, without epigenetic manipulation of the DNA of the mesenchymal stem cells. Further, the use of cell-free conditioned medium eliminates the possibility of cell fusion between the mesenchymal stem cells and hNSCs. Additionally, the use of serum-free medium containing well-defined components including recombinant mitogenic growth factors, preferably human recombinant mitogenic growth factors, prevents the exposure of mesenchymal stem cells to unknown chemical factors. Thus, the mesenchymal-derived neural stem cells made according to the methods of the invention meet the basic conditions required for generating clinical-grade therapeutic cells.

As used herein, the phrase “committed to the neural lineage” or “committed to neural lineage differentiation” refers to at least 85%, at least 90%, at least 95% or 100% of a population of mesenchymal-derived neural stem cells that is capable of differentiation into and only into neural cells.

The term “neural cells” as used herein refers to all neural-lineage cells including without limitation neurons, glial cells, astrocytes, and oligodendrocytes.

The methods of the invention comprise the step of culturing mesenchymal stem cells in a cell-free media conditioned by human neural stem cells (hNSCcm). In particular embodiments, conditioned medium is obtained by culturing human neural stem cells in a serum-free medium containing B27 or N2 and supplemented with mitogenic growth factors human bFGF (basic fibroblast growth factor) and human EGF (epidermal growth factor). In certain embodiments, the human neural stem cell culture medium further comprises human LIF (leukemia inhibitory factor). In certain alternative embodiments, the serum-free medium further optionally contains heparin (5 μg/ml, Sigma, St. Louis, Mo.), 2 mM L-glutamine and/or an antibiotic-antimycotic mixture (1:100, Invitrogen).

A mitogenic growth factor, as used herein, refers to a growth factor or a peptide thereof that has a growth or proliferative effect on neural stem cells. In certain embodiments, the mitogenic growth factors are human factors. In certain particular embodiments, the mitogenic growth factors are recombinant human factors.

In advantageous embodiments of the inventive methods, about half of the culture medium is collected every 2-3 days and the collected conditioned medium is passed through filter membranes with a pore size of 0.22 μm in diameter to remove any cells or cell debris present in the conditioned medium. Fresh medium is added to the human neural stem cells for continuous culture. In certain particular embodiments, the conditioned medium is collected every 2-3 days from the human neural stem cell culture as described herein for 50-60 days. In certain particular embodiments, the conditioned medium is continuously collected every 2-3 days from human neural stem cell culture for no more than 60 days in culture. As set forth in more detail herein, MeSCs cultured in the conditioned medium developed into mesenchymal-derived neural stem cells that are capable of differentiating into neural cells. In one aspect of the invention, the MeSCs are cultured in the hNSCcm and develop into the mesenchymal-derived neural stem cells. In certain particular embodiments, the hNSCcm is further supplemented with at least one human mitogenic growth factor. In certain embodiments, the mitogenic growth factor is human bFGF, EGF, LIF, PDGF, or FGF8 (fibroblast growth factor 8), preferably recombinantly-produced embodiments thereof. Recombinant human growth factors described herein are commercially available from, for example, R&D Systems Inc. (Minneapolis, Minn.) (catalog Nos. 233-FB for bFGF, 236-EG for EGF, and 220-BB for PDGF-BB), CHEMICON (Romona, Calif.) (catalog No. LIF1005 for LIF) and GenWay Biotech, Inc. (San Diego, Calif.) (catalog No. 10-783-79090 for FGF 8).

The invention also provides methods for treating a patient having a neurological disease, deficit, defect, deficiency or disorder, using the mesenchymal-derived neural stem cells as described herein. As used herein, the term “patient” refers to a mammal, preferably a human, and most preferably a human that has a neurological disease, deficit, defect, deficiency or disorder.

Advantageously, the invention allows neuroreplacement therapy using clinical-grade autologous mesenchymal-derived neural stem cells as described herein. While isolation of hNSCs from the brain tissue has been achieved, autologous transplants using the patient's neural cell source is not particularly feasible. Mesenchymal stem cells from bone marrow are a particularly good source of cells for generating mesenchymal-derived neural stem cells for autologous neuroreplacement therapy. Isolation techniques for mesenchymal cells are well established in the art and further described herein, and such techniques can be performed autologously.

As used herein, the term “neuroreplacement therapy” refers to a treatment or therapy to a patient an injection of the mesenchymal-derived neural stem cells of the invention. The mesenchymal-derived neural stem cells injected into the patient differentiate in a tissue-specific manner at the area of tissue damage and function in a manner that reduces the neurological deficits. Advantageously, the mesenchymal-derived neural stem cells can be injected at a distal site of the aged or injured neural tissues to minimize further damaging the aged or injured tissues in need of treatment or repair. In certain embodiments of the invention, the mesenchymal-derived neural stem cells injected into the patient at a distal site of the aged or injured neural tissues can migrate to the area of tissue damage, differentiate in a tissue-specific manner at the area of tissue damage and function in a manner that reduces the neurological deficit. The newly differentiated neural cells from the mesenchymal-derived neural stem cells replace the damaged neural tissues and can reverse or halt the course of neurodegeneration.

In addition to neuroreplacement therapy, the mesenchymal-derived neural stem cells of the invention may have neurotrophic effect to protect damaged or degenerated neuronal cells from dying and/or may stimulate neurogenesis of host neural stem cells when transplanted into the recipient's CNS.

Thus, according to the methods of this invention a patient's mesenchymal stem cells are isolated, treated as set forth herein, and transplanted back into the same patient to differentiate in a tissue-specific manner. Hence, the invention provides reagents (cells) and methods for treating neurological deficits without the problems associated with heterologous transplants from adult or fetal sources. Though less preferred, heterologous mesenchymal-derived neural stem cells prepared according to the method described herein and method of using such heterologous stem cells are within the scope of the invention.

As used herein, the terms “multipotent neural stem cells (MNSCs),” “neural stem cells (NSCs)” and “neural progenitor cells (NPCs)” refer to undifferentiated, multipotent cells of the CNS. Such terms are commonly used in the art, particularly the scientific literature. NSCs can differentiate into tissue-specific cell types, for example astrocytes, oligodendrocytes, and neurons when transplanted into the brain.

As used here, the term “mesenchymal-derived neural stem cells” refers to stem cells that are derived from mesenchymal cells, not derived from neural tissue or neural progenitor cells, and are capable of differentiating into cells of neural lineage. The mesenchymal-derived neural stem cells of the invention are similar to NSC in that they are capable of differentiating into neural cells. They are different, however, in that mesenchymal-derived neural stem cells of the invention are not derived from neural progenitor cells or neural-lineage cells. This invention encompasses both heterologous and autologous mesenchymal-derived neural stem cells, pharmaceutical compositions and cellular preparations containing the same, and methods of making and using the same. In certain advantageous embodiments, this invention provides autologous mesenchymal-derived neural stem cells, pharmaceutical compositions and cellular preparations containing the same, and methods of making and using the same.

The mesenchymal-derived neural stem cells of the invention can form a cluster of two or more cells. The clusters can comprise the progeny of a single or multiple mesenchymal-derived neural stem cells. In certain particular embodiments, the clusters are about or less than 0.1 mm in diameter and may contain 100 to 500 cells. In certain other embodiments, the clusters are disrupted to facilitate growth when the clusters reach 0.5 mm in diameter.

In certain embodiments, the mesenchymal-derived neural stem cells spontaneously differentiate into neural cells in a basal medium. In particular embodiments, the basal medium is further optionally supplemented with B27 or N2. B27 and N2 supplements are commercially available from, for example Invitrogen (catalog Nos. 17504044 for B27 and 17502048 for N2). The B27 supplement contains biotin, L-carnitine, corticosterone, ethanolamine, D(+)-galactose, glutathione (reduced), linoleic acid, linolenic acid, progesterone, putrescine, retinyl acetate, selenium, T3 (Triodo-1-Thyronine), DL-α-trocopherol (Vitamin E), DL-α-trocopherol acetate, bovine albumin, catalase, insulin, superoxide dismutase, and transferrin (Brewer et al., 1993, J. Neuroscience Res. 35:567-576). The N2 supplement contains human transferrin, insulin, progesterone, putrescine, and selenite. The B27 and N2 are used in the medium at concentrations of 1:50 and 1:100 according to manufacturer's recommendation for neural differentiation.

Basal medium suitable for use in the current invention comprises a serum-free, mitogen-free medium commonly used for spontaneous differentiation of neural stem cells into neural cells. Non-limiting examples of basal medium includes serum-free medium commercially-available from Invitrogen, for example, Neurobasal™ Medium, catalog No. 10888022.

The mesenchymal-derived neural stem cells of the invention express early or embryonic neural markers, similar to neural stem cells or neural progenitor cells. Non-limiting exemplary early neural markers include neural progenitor marker stage-specific embryonic antigen 4 (SSEA4; see, for example, Sato et al., 2007, Biochem Biophys Res Commun. 364:838-43, Rao et al., 2007, Methods Mol Biol. 407:51-61), BF1 (for example, GenBank Accession No. BC050072, SEQ ID NO: 11), Nurr1 (nuclear receptor related protein 1, for example, GenBank Accession No. AB019433, SEQ ID NO: 12), NeuroD (for example U50822, SEQ ID NO: 13), POU domain transcription factor 4 (also known as OCT4, for example BC117435, SEQ ID NO:14), Nanog (for example AB093576, SEQ ID NO: 15), human paired box gene 6 (Pax6, for example M93650, SEQ ID NO: 16), Sox1 (for example Y13436, SEQ ID NO: 17), or Nestin (for example NM_{—}006617, SEQ ID NO: 18). Expression of one or more early neural markers is indicative of cell fate alteration of the mesenchymal-derived neural stem cells from a mesenchymal cell development pathway to a neural cell development pathway. In certain embodiments, the mesenchymal-derived neural stem cells express early neural markers but no longer express fibronectin, a marker typically for mesenchymal stem cell.

The mesenchymal-derived neural stem cells of the invention spontaneously differentiate into neural cells without co-culturing with differentiating or differentiated neural stem cells. In certain embodiments, the mesenchymal-derived neural stem cells differentiate into neural cells by culturing in the basal medium for 5-7 days. The cells can be allowed to further differentiate in the basal medium for 7-10 days, in the presence or absence of neurotrophic factors including without limitation β-NGF (beta nerve growth factor), BDNF (brain-derived neurotrophic factor), and GDNF (Glial cell line-derived neurotrophic factor). In certain embodiments, the neurotrophic factors are human, preferably recombinant human factors. Recombinant human neurotrophic factors are commercially available from, for example, R&D System Inc. (Minneapolis, Minn.) (catalog Nos. 256-GF for NGF, 248-BD/CF for BDNF, and 212-GD for GDNF).

In certain particular embodiments, the neurons differentiated from the mesenchymal-derived neural stem cells of the invention exhibit action membrane potential similar to mature neurons derived from neural stem cells. In certain further embodiments, the neural cells derived from the mesenchymal-derived neural stem cells express neural markers including without limitation beta III-tubulin (for example, GenBank accession No. NM_{—}006086, SEQ ID NO:19), NG2 (NM_{—}001897, SEQ ID NO:20), neuron nuclear antigen (NeuN, Sarnat et al., 1997, Brain Development 20:88-94), microtubule-associated protein 2 (MAP2, for example, U01828, SEQ ID NO:21), or glial fibrillary acidic protein (GFAP, for example, AF419299, SEQ ID NO:22). In certain embodiments, the neural cells derived from the mesenchymal-derived neural stem cells are immunopositive for gamma-aminobutyric acid (GABA), galactocerebroside (GalC, myelin-specific glycolipid galactocerebroside) or glutamate.

In certain embodiments, the expression of embryonic stem cell markers, neural stem cells markers or mature neural markers as described herein can be detected by immunostaining using commercially available antibodies specific for said proteins. The following provides non-limiting examples of commercial sources for the antibodies: anti-OCT 4 antibody (catalog No. MAB4305 from CHEMICON, Romona, Calif.), anti-Sox-1 antibody (catalog No. AB5768 from CHEMICON), anti-SSEA4 antibody (catalog No. MAB4304 from CHEMICON), anti-Nestin antibody (catalog Nos. MAB353 or AB5922 from CHEMICON), anti-GFAP antibody (catalog No. MAB360 from CHEMICON, catalog No. ab929 from Abcam, Cambridge, Mass.), anti-NG2 antibody (catalog No. AB5320 from CHEMICON), anti-galactocerebroside antibody (catalog No. AB142 from CHEMICON), anti-MAP2 antibodies (catalog No. MAB3418 from CHEMICON), anti-Nanog (catalog No. ab21603 from Abcam), anti-Pax-6 antibody (catalog No. sc-7750 from Santa Cruz Biotechnology, Santa Cruz, Calif.), anti-BF1 antibody (catalog No. sc-18583 from Santa Cruz Biotechnology), anti-beta III tubulin antibody (catalog No. T8660 from Sigma, catalog No. MRB-435P from COVANCE, CA), anti-glutamate antibody (catalog No. G6642 from Sigma), and anti-γ-Aminobutyric Acid (GABA) (catalog No. A0310 from Sigma).

As used herein, the term “effective amount” or “therapeutically effective amount” refers to the amount of reagents or factors used to support the desired activity. In the case of mesenchymal-derived neural stem cells prepared and delivered according to the invention, an effective amount is an amount necessary to support an observable level of one or more biological activities of mesenchymal-derived neural stem cells as set forth herein. It is within the ability of an ordinary skilled artisan or physician to determine an effective amount of mesenchymal-derived neural stem cells for treatment of a given neurological disease, deficit, deficiency of disorder. Regarding the method of making mesenchymal-derived neural stem cells from mesenchymal stem cells, the effective amount of mitogenic growth factors bFGF, EGF, or LIF in the conditioned medium can be between about 3-40 or 3-5 ng/ml, 3-40 or 7-10 ng/ml, and 1-20 or 1-3 ng/ml, respectively. Regarding differentiation of mesenchymal-derived neural stem cells to neural cells, the effective amount of neurotrophic factors NGF, BDNF, or GDNF in the basal medium can be about 5-50 or 5 ng/ml, 5-50 or 10 ng/ml, and 10-100 or 25 ng/ml, respectively.

An “effective period” as used herein refers to the time period necessary for the reagents and cells of the invention to accomplish their specified activities. For example, mesenchymal stem cells (MeSCs) can be allowed to develop into mesenchymal-derived neural stem cells of the invention by culturing the MeSCs in a cell-free human neural stem cell conditioned medium for an effective period, which can be about 3-4 weeks, preferably about 4 weeks. Further, mesenchymal-derived neural stem cells can be cultured in a basal medium for a period effective to allow mesenchymal-derived neural stem cells to differentiate into neural cells. The effective period for differentiating the mesenchymal-derived neural stem cells of the invention into neural cells can be about 5-7 days, 5-10 days, preferably about 6 days. Further, mesenchymal-derived neural stem cells of the invention can be contacted before or during differentiation in the basal medium with at least one neurotrophic factor such as NGF, BDNF, or GDNF, or combinations thereof, for a period effective to allow differentiation of neural cells.

As used herein, “ameliorating the effects caused by age, physical and biological trauma and degenerative disease” and the like refers to the relief or diminution of the detrimental effects of damaged or degenerated tissue by way of use or administration of an effective amount of the mesenchymal-derived neural stem cells of the invention, or pharmaceutical preparations thereof, to the affected, damaged or degenerated tissue, wherein the mesenchymal-derived neural stem cells can differentiate in a manner appropriate for the host tissue and enable the replacement of damaged cells, repair of damaged tissue and reduction of structural or functional loss. Such amelioration is affected through the administration to the mammal/patient of an effective amount of the mesenchymal-derived neural stem cells or pharmaceutical compositions or cellular preparations thereof of the invention.

The invention also provides cellular preparations and pharmaceutical compositions of the mesenchymal-derived neural stem cells of the invention and methods of delivery into the brain and other tissues thereof. The cellular preparations and pharmaceutical compositions of the invention may contain formulation materials such as pharmaceutically acceptable carriers, diluents, excipients for modifying, maintaining, or preserving, in a manner that does not hinder neural differentiation of the mesenchymal-derived neural stem cells of the invention, for example, pH, osmolarity, viscosity, clarity, color, isotonicity, odor, sterility, stability, rate of dissolution or release, adsorption, or penetration of the composition, as well as proliferation, migration and differentiation capacity of the mesenchymal-derived neural stem cells of the invention. Suitable formulation materials include, but are not limited to, amino acids (such as glycine, glutamine, asparagine, arginine, or lysine), antimicrobial compounds, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, betacyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; Triton; trimethamine; lecithin; cholesterol or tyloxapal), stability enhancing agents (such as sucrose or sorbitol), tonicity enhancing agents (such as alkali metal halides—preferably sodium or potassium chloride—or mannitol sorbitol), delivery vehicles, diluents, excipients and/or pharmaceutical adjuvants. See REMINGTON'S PHARMACEUTICAL SCIENCES (18th Ed., A. R. Gennaro, ed., Mack Publishing Company 1990).

The primary vehicle or carrier in a pharmaceutical composition may be either aqueous or non-aqueous in nature. For example, a suitable vehicle or carrier for injection may be physiological saline solution, or artificial cerebrospinal fluid. Optimal pharmaceutical compositions can be determined by a skilled artisan depending upon, for example, the intended route of administration, delivery format, desired dosage and recipient tissue. See, e.g., REMINGTON'S PHARMACEUTICAL SCIENCES, supra. Such compositions may influence the physical state, stability, and effectiveness of the composition.

The invention also provides kits comprising the cell-free human neural stem cell conditioned medium of the invention for developing the mesenchymal-derived neural stem cells from mesenchymal stem cells and instructions for use thereof. In certain particular embodiments, the kits further comprise at least one mitogenic growth factor including without limitation bFGF, EGF, LIF, PDGF, and FGF8. In other embodiments, the kits also comprise reagents for the preparation of Ficoll-Hypaque gradients for isolating mesenchymal stem cells from bone marrow. In yet other particular embodiments, the kits further comprise a basal medium for use in differentiating the mesenchymal-derived neural stem cells to neural cells in vitro.

The invention also provides methods of delivery and transplantation of the mesenchymal-derived neural stem cells of the invention to ameliorate the effects of aging, physical and biological trauma and degenerative disease on the brain or central nervous system of a mammal. It is well recognized in the art that transplantation of tissue into the CNS offers the potential for treatment of neurodegenerative disorders and CNS damage due to injury. Transplantation of new cells into the damaged CNS has the potential to repair damaged neural circuitries and provide neurotransmitters thereby restoring neurological function. As disclosed herein, the invention provides methods for generating mesenchymal-derived neural stem cells adapted for proliferation, migration and differentiation in mammalian tissues when introduced thereto. The use of mesenchymal-derived neural stem cells in the treatment of neurological disorders and CNS damage can be demonstrated by the use of established animal models known in the art.

There are significant advantages in the method of delivery to the brain of the mesenchymal-derived neural stem cells compared to the prior art. In a non-limiting example, the mesenchymal-derived neural stem cells of the invention are transplanted intraventricularly. The mesenchymal-derived neural stem cells of the invention can be transplanted into a patient as individual cells or in the form of clusters via a surgical procedure or injection using a syringe large enough to leave the clusters substantially intact. In certain particular embodiments, the mesenchymal-derived neural stem cells are transplanted as individual cells at sufficient numbers to be efficacious in ameliorating the neurological disease, deficit, defect, deficiency or disorder. Another benefit of intraventricular injection is less tissue destruction, resulting in less localized recruitment of immune cells by the host. This can be shown by a demonstration of lack of ventricular distortion, tumor formation, and increased host astrocyte staining without any immunosuppression.

There are examples in the art of intra-tissue injection (brain) of dissociated and partially differentiated NSCs (see, e.g., Benninger et al., 2000, Brain Pathol. 10: 330-341; Blakemore et al., 2000, Cell Transplant. 9: 289-294; Rosser et al., 2000, Eur. J. Neurosci. 12: 2405-2413; Rubio et al., 2000, Mol. Cell. Neurosci. 16: 1-13). Further, dissociation of NSC neurospheres is known to cause immediate senescence of NSCs and increase the vulnerability of NSCs in culture. See, e.g., Svendsen et al., 1998, J. Neurosci. Methods 85: 141-152. Some aspects of the instant invention preferentially employ injection of clusters of 100 or more cells, which can be easily removed and isolated from cell culture and used for transplantation. The mesenchymal-derived neural stem cells of the invention can migrate and differentiate appropriately when the clusters of cells are transplanted in non-cluster form as well. The cells in non-cluster form are cells that grow as clusters in culture and are dispersed into individual cells by gentle mechanical methods before transplantation. Gentle mechanical methods for disrupting clusters of cells include without limitation triturating the clusters through a fire-polished glass pipette. The clinical choice to use cluster or individual cells in transplantation will depend on disease conditions and route of injection. For local intra-tissue injection, dissociation of the clusters gently by mechanical methods may be necessary; for cerebroventrical injection, dissociation of the clusters may not be needed. Cerebroventricular transplantation with cell clusters can be a useful therapeutic paradigm for diseases that require brain-wide cell replacement, such as Alzheimer's diseases (AD), in which significant neuronal loss appears in many brain regions. The use of a classical neural transplantation approach, i.e., injection of individual cell suspension into the proximity of the site of the lesion or into its target area, is suitable for stroke, brain damage, trauma, and Parkinson's disease. Such clinical choices are within the skill of the ordinarily-skilled physician, as is a determination of a suitable number of individual cells or clusters of cells to be injected into a patient in need thereof.

As provided by this invention, intraventricular transplantation provides an advantageous and alternative route to the site-specific injection taught in the prior art. Using intraventricular transplantation, grafted cells can gain access to a plurality of CNS structures by cerebral spinal fluid (CSF) flow, and transplantation of mesenchymal-derived neural stem cells of the invention in cluster form can prevent premature differentiation at inappropriate anatomical sites in the brain and central nervous system. In other neurological sites, for example the eye, intraocular administration of clusters of two or more cells, such as into the vitreous fluid, permits the mesenchymal-derived neural stem cells of the invention to migrate to the area of degeneration or injury and differentiate appropriately.

In certain alternative embodiments, the mesenchymal-derived neural stem cells are injected into a mammal in need thereof by intravenous injection. In some neurological conditions, such as stroke or brain injuries, the blood-brain barrier is compromised, which allows intravenously injected mesenchymal-derived neural stem cells to cross the blood-brain barrier.

It is an advantage of the autologous, mesenchymally-derived neural stem cells of the invention that recipients do not need to be immunosuppressed as do nonautologous transplantation recipients. Nevertheless, recipients of the mesenchymal-derived neural stem cells of the invention can be immunosuppressed, either through the use of immunosuppressive drugs such as cyclosporin, or through local immunosuppression strategies employing locally applied immunosuppressants. Such immunosuppression may not be necessary in certain immunoprivileged tissues, for example, brain and eye tissues, or other non-immunoprivileged tissues due to the advantages of autologous transplantation. In certain embodiments, the delivery method of the invention can cause less localized tissue damage to the site of cell damage or malfunction than existing methods of delivery.

Functional integration of the graft into the host's neural tissue can be assessed by examining the effectiveness of grafts on restoring various functions, including but not limited to tests for endocrine, motor, cognitive and sensory functions. Useful motor tests include tests that quantify rotational movement away from the degenerated side of the brain, and tests that quantify slowness of movement, balance, coordination, akinesia or lack of movement, rigidity and tremors. Cognitive tests include tests of the ability to perform everyday tasks, as well as various memory tests, including maze performance such as the Morris water maze performance.

Without being restricted to any particular theory for the mechanism of action of the cells and methods of the invention, at least two explanations for the beneficial effects of mesenchymal-derived neural stem cell transplantation to cognitive function of the host brain can be envisioned. One is replacement and/or augmentation. Neuronal circuits can be replaced or augmented by the mesenchymal-derived neural stem cell-derived neurons. In other tissues, cells and cell structures can also be replaced or augmented by mesenchymal-derived neural stem cell-derived cells appropriate for that tissue. Another alternative explanation for the beneficial effects of administering the mesenchymal-derived neural stem cells of the invention is the neurotrophic action of factors released from the transplanted mesenchymal-derived neural stem cells.

As used herein, the phrase “a neurological disease, deficit, defect, deficiency, or disorder” refers to decreased or loss of brain function or neural degeneration, the cause of which can be aging, infection, trauma, genetic or epigenetic changes.

Mesenchymal-derived neural stem cells can be used to treat neurological deficits caused by a wide variety of diseases, disorders, and injuries, at least in part due to the discovery by the instant inventors that mesenchymal-derived neural stem cells can migrate and differentiate in brain. These insults include, but are not limited to, the following:

### Neurodegenerative Diseases

One cause of impaired or improper brain function is neurodegenerative disease. In recent years neurodegenerative disease has become an important concern due to an expanding elderly population that is at greatest risk for these disorders. In fact, neurodegeneration also encompasses many conditions and diseases, age-related or not, that result in neuronal loss. These conditions include CNS trauma, such as stroke and epilepsy, as well as diseases that result in neuronal loss, including amyotrophic lateral sclerosis and cerebral palsy. The inventive mesenchymal-derived neural stem cells can be used to counteract neurodegeneration as the results of the following diseases or conditions: Alzheimer's disease (AD), Parkinson's disease (PD), Huntington's disease (HD), Pick's disease, progressive supranuclear palsy (PSP), striatonigral degeneration, cortico-basal degeneration, Parkinson-ALS-dementia complex, olivopontocerebellar atrophy (OPCA; including a heritable form), Gerstmann-Straussler-Scheinker syndrome, Leigh's disease, infantile necrotizing encephalomyelopathy, Hunter's disease, Wilson's disease, multiple sclerosis (MS), mucopolysaccharidosis, various leukodystrophies (such as Krabbe's disease, Pelizaeus-Merzbacher disease, and the like), amaurotic (familial) idiocy, Kuf's disease, Spielmeyer-Vogt disease, Tay Sachs disease, Batten disease, Jansky-Bielschowsky disease, Reye's disease, cerebral ataxia, chronic alcoholism, beriberi, Hallervorden-Spatz syndrome, and cerebellar degeneration.

There are a variety of brain diseases that impair motor or cognitive function. Degeneration in the basal ganglia can lead to diseases with cognitive and motor symptoms, depending on the exact location of the degeneration. The basal ganglia consists of many separate regions, including the striatum (which consists of the caudate and putamen), the globus pallidus, the substantia nigra, substantia innominata, ventral pallidum, nucleus basalis of Meynert, ventral tegmental area and the subthalamic nucleus.

Motor deficits are a common result of degeneration in the basal ganglia. Huntington's Chorea is associated with degeneration of neurons in the striatum, which leads to involuntary jerking movements in the host. Degeneration of a small region called the subthalamic nucleus is associated with violent flinging movements of the extremities in a condition called ballismus, while degeneration in the putamen and globus pallidus is associated with a condition of slow writhing movements or athetosis. In Parkinson's disease, degeneration is seen in another area of the basal ganglia, the substantia nigra par compacta. This area normally sends dopaminergic connections to the dorsal striatum, which are important in regulating movement. Therapy for Parkinson's disease has centered upon restoring dopaminergic activity to this circuit, which can be accomplished by transplantation of mesenchymal-derived neural stem cells to this region of the brain according to the instant invention.

Alzheimer's disease patients exhibit a profound cellular degeneration of the forebrain and cerebral cortex. Further, a localized area of the basal ganglia, the nucleus basalis of Meynert, appears to be selectively degenerated. This nucleus normally sends cholinergic projections to the cerebral cortex that are thought to participate in cognitive functions including memory.

Additionally, due to the generally low proliferation rate of mammalian NSCs, there is a correlation between advancing age and impaired brain function even in the absence of specific neurodegenerative disease or physical or biological brain trauma. The invention provides methods for counteracting impaired brain function due to advancing age through the addition of mesenchymal-derived neural stem cells capable of proliferation, migration and differentiation in mammalian brain when introduced thereto.

Physical or Biological Trauma and Neurotoxic injuries to the Central Nervous System

Physical trauma and biological trauma are also causes of impaired or improper brain function. The term “physical trauma” denotes brain cell damage due to external sources such as blunt head trauma, severe concussion and the like. Such physical trauma can be localized or general depending on the source and severity of the trauma. The term “biological trauma” denotes any acute brain injury that has its origin in a biological process, for example, stroke, aneurysm, epilepsy, brain tumor, hypoxia and the like.

Traumatic injuries to the CNS that can be treated according to the methods of the invention include gunshot wounds, injuries caused by blunt force, injuries caused by penetration injuries (e.g., stab wounds), injuries caused in the course of a surgical procedure (e.g., to remove a tumor or abscess from the CNS or to treat epilepsy), poisoning (e.g., with MPTP or carbon monoxide), shaken-baby syndrome, adverse reactions to medication (including idiosyncratic reactions), drug overdose (e.g., from amphetamines), and post-traumatic encephalopathy.

### Ischemia

Stroke is the rapidly developing loss of brain functions due to a disturbance in the blood vessels supplying blood to the brain. Any disruption of blood flow or oxygen delivery to the nervous system can injure or kill cells, including neurons and glial cells, therein. Strokes can occur as a result of ischemia caused by thrombosis or embolism or due to a hemorrhage. The brain tissue affected by ischemia in a stroke patent exhibits a spectrum of damages, from irreversible damage to injuries that could potentially recover.

These injuries can be treated according to the methods of the invention and include injuries caused by a stroke (including a global stroke (as may result from cardiac arrest, arrhythmia, or myocardial infarction) or a focal stroke (as may result from a thrombus, embolus, hemorrhage, or other arterial blockage)), anoxia, hypoxia, partial drowning, myoclonus, severe smoke inhalation, dystonias (including heritable dystonias), and acquired hydrocephalus.

### Developmental Disorders

Developmental disorders that can be treated according to the methods of the invention include schizophrenia, certain forms of severe mental retardation, cerebral palsy (whether caused by infection, anoxia, premature birth, blood type incompatibility: etc. and whether manifest as blindness, deafness, retardation, motor skill deficit, etc.), congenital hydrocephalus, metabolic disorders affecting the CNS, severe autism, Down Syndrome, LHRH/hypothalamic disorder, and spina bifida. In certain particular embodiments, mesenchymal-derived neural stem cells obtained from genetically matched healthy donors prepared as described herein are used for treating hereditary neurological developmental disorders.

### Injuries and Diseases of the Spinal Cord

Injuries to or diseases affecting the spinal cord can also be treated according to the methods of the invention. Such injuries or diseases include post-polio syndrome, amyotrophic lateral sclerosis, nonspecified spinal degeneration, traumatic injury (such as those caused by automobile or sporting accidents), including any injury that crushes, partially severs, completely severs, or otherwise adversely affects the function of cells in the spinal cord), injuries caused by surgery to the spinal cord (e.g., to remove a tumor), anterior horn cell disease, and paralytic diseases.

### Demyelinating or Autoimmune Disorders

Neurological deficits caused by demyelination or an autoimmune response can be treated according to the methods of the invention. Such deficits can be caused by multiple sclerosis, or lupus.

### Infectious or Inflammatory Diseases

Neurological deficits caused by an infection or inflammatory disease can be treated according to the methods of the invention. Infections or inflammatory diseases that can cause treatable deficits include Creutzfeldt-Jacob disease and other slow virus infectious diseases, AIDS encephalopathy, post-encephalitic Parkinsonism, viral encephalitis, bacterial meningitis and meningitis caused by other organisms, phlebitis and thrombophlebitis of intracranial venious sinuses, syphilitic Parkinsonism, and tuberculosis of the CNS.

In addition to the deficits, diseases and disorders explicitly set forth above, those of ordinary skill in the art are well able to recognize neurological deficits, regardless of their cause, and to apply the methods of the invention to treat patients who have such deficits. In addition to the conditions listed above, that are amenable to treatment with the methods described herein, neurological deficits can be caused by myasthenia gravis, various dementias, numerous parasitic diseases, and epilepsy. Further, alleviation of age-related memory loss is also an object of the invention. The methods of the invention can be readily applied to alleviate neurological deficits caused by these and other diseases, disorders, or injuries.

Mesenchymal-derived neural stem cells of the invention can serve as an alternative to NSCs for potential therapeutic uses utilizing the methods of the invention, which exploit the capacity of neural stem cell conditioned medium to alter the normal differentiation fate of MeSCs. These cells are important in the neuroreplacement therapy field because their production permits autologous transplantation; since the cells originate from the patient, there are no ethical barriers and reduced or absent immunorejection issues raised by using the cells and methods of this invention.

The following examples are presented in order to more fully illustrate the preferred embodiments of the invention. They should in no way be construed, however, as limiting the scope of the invention, as defined by the appended claims.

### EXAMPLES

### Example 1

### Preparation and Collection of hNSCcm

Human NSCs (hNSCs) obtained from Biowhittaker (Walkersville, Md.) were cultured and expanded in a serum-free culture media comprising bFGF, EGF and LIF (leukemia inhibitory factor) in the presence of B27. hNSC cultured in this medium grew into neurospheroids, which were routinely sectioned into quarters every 2 weeks and fed by replacing 50% of the medium every 2-3 days to facilitate optimal growth of the cells. The replaced medium from hNSC cultures, which is referred to as human neural stem cell conditioned medium (hNSCcm), was filtered through a membrane with a pore size of 0.22 μm in diameter, and stored at −20° C. for later use.

### Example 2

### Mononuclear Cells Isolated from Bone Marrow Tissues and Cultured in hNSCcm Developed into Clinical Grade Mesenchymal-Derived Neural Stem Cells that Expressed Early Neural Markers

Fresh human bone marrow tissues were obtained from ALLCells Inc. (Emeryville, Calif.) and BioWhittaker (Walkersville, Md.). Mononuclear bone marrow cells were isolated by Ficoll density centrifugation and plastic adherence selection. Briefly, the bone marrow tissue was diluted in iso-osmolar solution of Ca²/Mg²⁺-free Dulbecco's phosphate buffered saline (D-PBS) (Invitrogen) to a concentration of 5×10⁶ mononuclear cells per ml, over-layered on a column of Ficoll-Hypaque (Amersham Biosciences, Piscataway, N.J.) and centrifuged at 400×g for 30 minutes at 18° C. Monolayer bone marrow cells were aspirated from the interface, washed in D-PBS, and cultivated in a T-75 flask at a concentration of 1×10⁴/cm² in DMEM (Invitrogen) containing an antibiotic antimycotic mixture (1:100) and 10% FBS (Stem Cell Technologies, Vancouver, BC) at 37° C. in a 5% CO₂ humidified incubator. Monolayer bone marrow cells contain a mixture of cell types including mesenchymal stem cells, red blood cells, and hematopoietic progenitor cells. Mesenchymal stem cells, but not red blood cells or hematopoietic progenitor cells, are adherent to tissue culture plastic within 24 to 48 hours. Thus, the medium of the monolayer cell culture was changed every three days to remove those suspending non-mesenchymal stem cells. The monolayer human mesenchymal stem cells (hMeSCs) so obtained became mesenchymal-derived neural stem cells capable of neural induction by growing in hNSCcm as described below.

The mesenchymal cells grown as monolayer culture were expanded for 6-8 passages and trypsinized by 0.05% trypsin and 0.53 mM EDTA at room temperature for 5 min, washed, suspended, and seeded in hNSCcm at a concentration of 1×10⁵/cm² in the chamber slides coated with 0.01% poly-L-lysine (Sigma). hNSCcm contained complex soluble factors released by proliferating hNSCs, which promote neural lineage commitment of hMeSCs. Twenty-one days after cultured in hNSCcm, expression of early neural markers in these mesenchymal-derived neural stem cells was examined by RT-PCR and/or fluorescence immunocytochemistry.

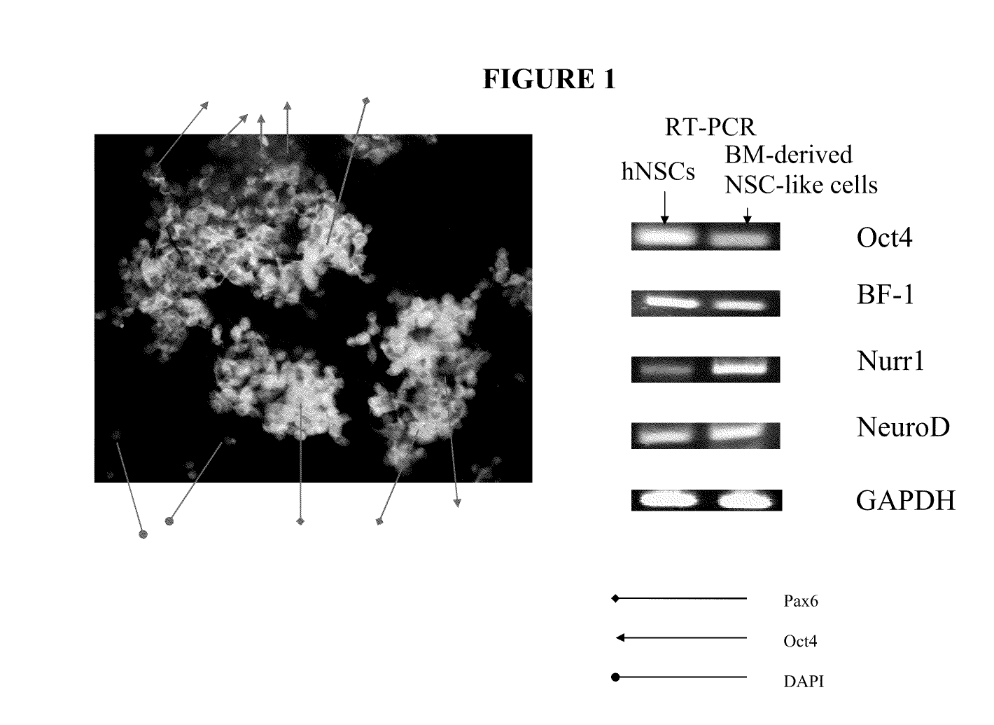

When cultured in hNSCcm in chamber slides for about 20 days, these hMeSCs changed from a monolayer to a mixture of a monolayer and overlying cell aggregates. The cell aggregates had a doubling time of 8-12 hours. These cells tended to grow as neurosphere-like structures and lost the fibroblast-like phenotypes of typical hMeSCs. Additionally, RT-PCR and immunocytochemistry studies demonstrated that hMeSCs grown in hNSCcm expressed a series of embryonic and/or neural stem cell markers such as SSEA4 and OCT4, and neural progenitor markers such as Nanog, Pax6, Nurr1, Sox1, BF1, NeuroD and Nestin. Exemplary results of expression of early neural markers are shown in FIG. 1. The gene expression pattern and the neurosphere-like structures of the cells suggested that these hMeSCs have switched from an adult program to an embryonic-like program when cultured in the hNSCcm. Thus, hMeSCs grown in the conditioned medium of human neural stem cells became mesenchymal-derived neural stem cells, similar to hNSC isolated from fetal brains that are capable of expressing embryonic neural markers. The sequences of RT-PCR primer pairs used in this study are shown below: for OCT4 detection, 5′ GTA TTC AGC CAA ACG ACC ATC 3′ (SEQ ID NO:1), 5′ GTT CGC TTT CTC TTT CGG GC 3′ (SEQ ID NO:2); for BF1 detection, 5′ ACT CAA AAC TCG CTG GGC AAC 3′ (SEQ ID NO:3), 5′CGT GGG GGA AAA AGT AAC TGG 3′ (SEQ ID NO:4); for Nurr1 detection, 5′CGA TGC CTT GTG TTC AGG CGC AG 3′ (SEQ ID NO:5), 5′ AGC CTT TGC AGC CCT CAC AGG TG 3′ (SEQ ID NO:6); for NeuroD detection, 5′CAG AAC CAG GAC ATG CCC 3′ (SEQ ID NO:7), 5′ ATC AAA GGA AGG GCT GGT G 3′ (SEQ ID NO:8); and for GAPDH control, 5′ ACC ACA GTC CAT GCC ATC AC 3′ (SEQ ID NO:9), 5′ TCC ACC ACC CTG TTG CTG TA 3′ (SEQ ID NO:10).

### Example 3

### Mesenchymal-Derived Neural Stem Cells Cultured in Basal Medium Spontaneously Developed into Neuronal and Glial Cells and Expressed Mature Neural Markers

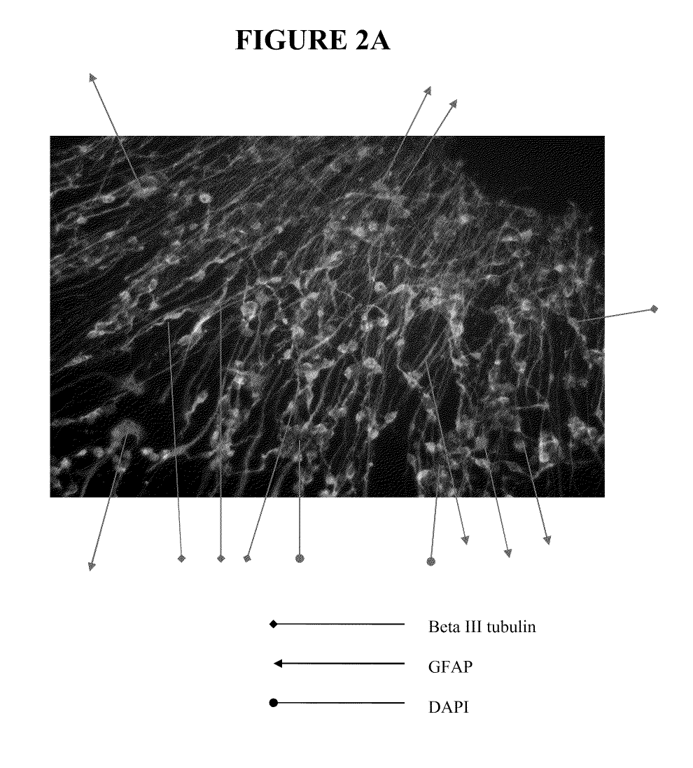

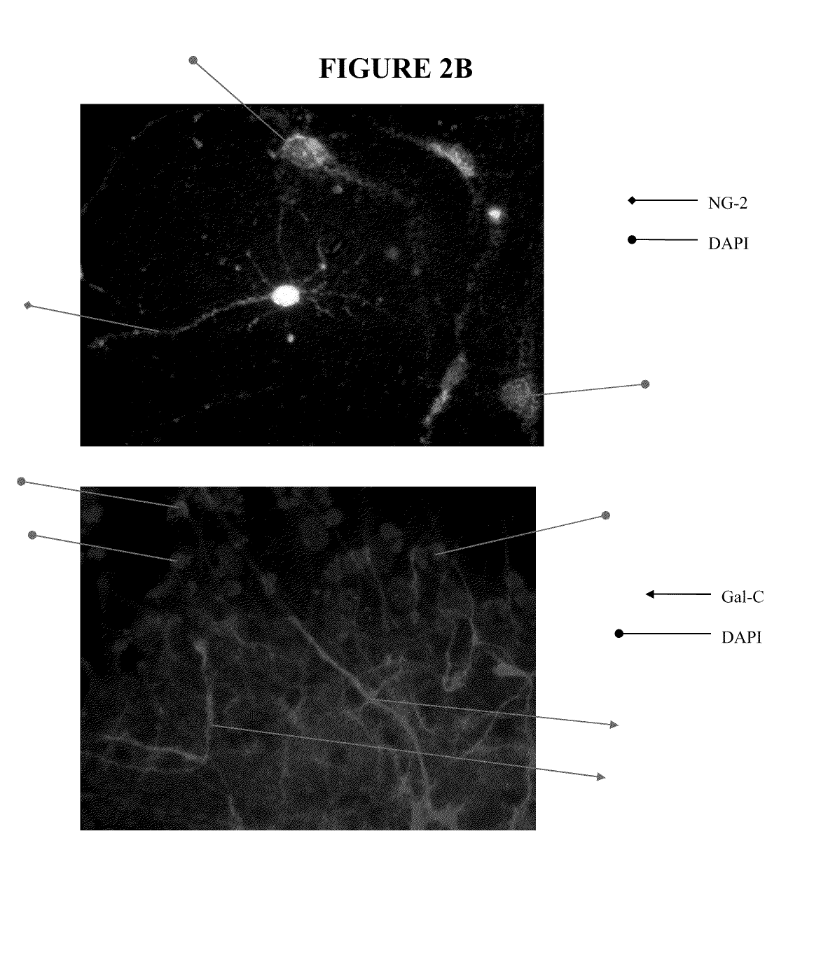

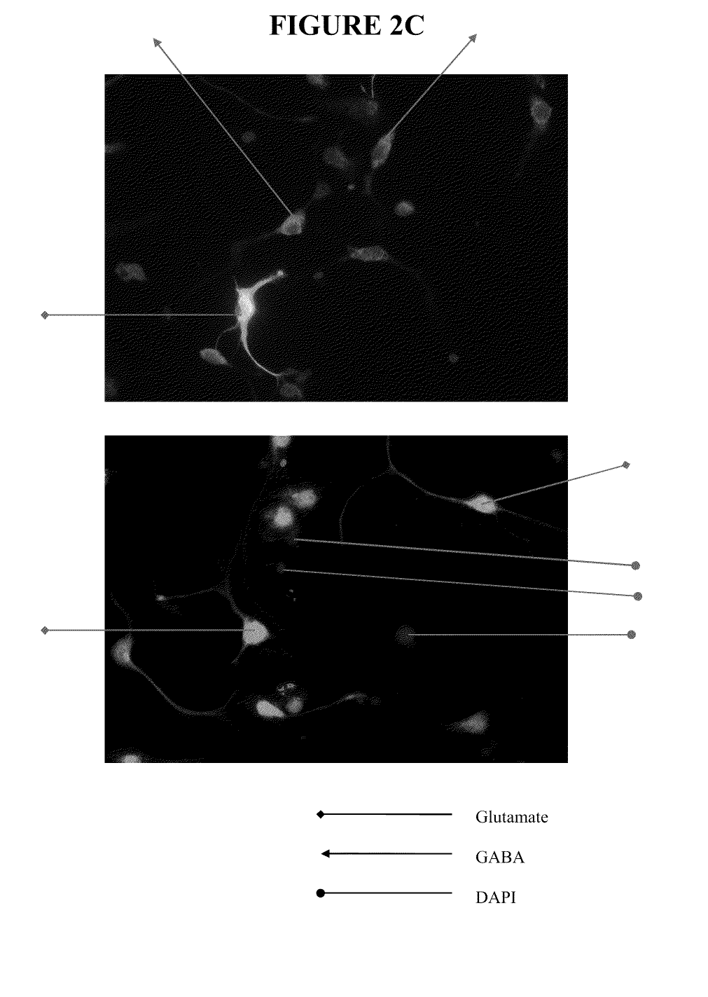

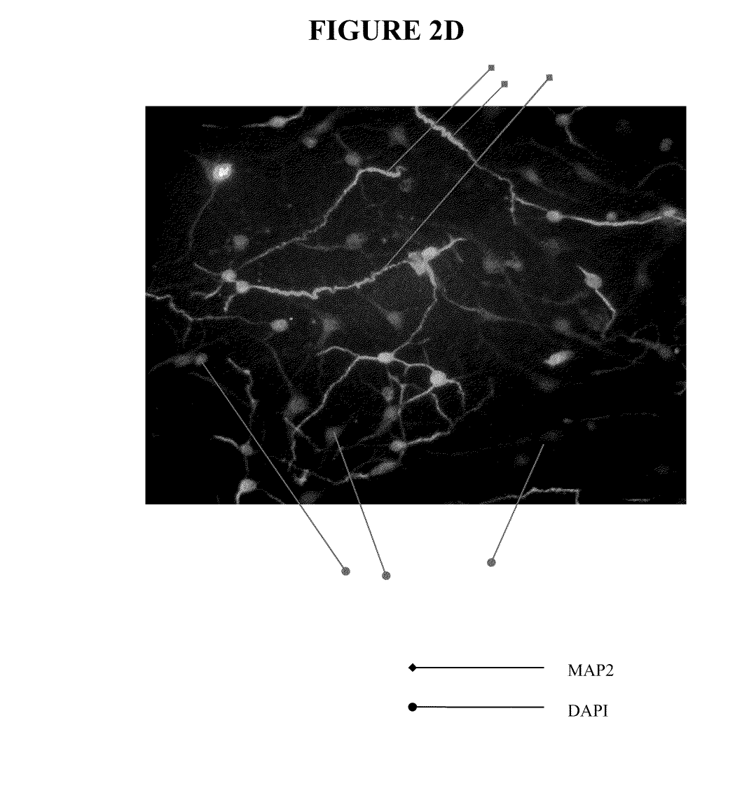

The capacity of mesenchymal-derived neural stem cells of the invention to differentiate into neural cells was further verified immunocytochemically by detecting the expression of mature neural markers. In basal medium supplemented with B27 or N2, which contains no inductive or promoting reagents for neural differentiation, hNSCs isolated from fetal brains spontaneously developed into neuronal and glial cells and expressed long lasting neural markers. In this experiment, mesenchymal-derived neural stem cells cultured in the hNSCcm as described above were grown in basal medium supplemented with B27. After five to seven days of culture in basal medium, about 40% of the cells expressed neuronal marker beta III-tubulin, about 60% of the cells expressed glial cell marker glial fibrillary acidic protein (GFAP), and oligo-dendrocyte markers NG2 and GalC as shown by immuno-staining (shown in FIGS. 2A and 2B). Upon further differentiation in the basal medium for 7-10 days in the presence of B27 and NGF, neural cells differentiated from mesenchymal-derived neural stem cells expressed additional mature neuronal cell markers including gamma-aminobutyric acid (GABA), glutamate, and microtubule-associated protein 2 (MAP2) (shown in FIGS. 2C and 2D). Once differentiated, the cells stopped proliferating. Although the axonal or dendritic processes of beta III-tubulin immunopositive cells were shorter than those found in differentiated hNSCs, the general morphology of the cells in both hNSC- and mesenchymal-derived neural stem cell-differentiated cultures was similar. These results supported the conclusion that the newly-generated cells from hMeSCs were indeed mesenchymal-derived neural stem cells because they behaved very much like the hNSCs isolated from fetal brain (Ma et al., 36^{th} Annual Meeting in Atlanta, Ga., October, 2006).

These mesenchymal-derived neural stem cells can be maintained in a proliferating state, expanded repeatedly (up to 30 passages to date) or stored in liquid nitrogen (storage medium: 10% DMSO in 1:1 of hNSCcm: culture medium for hNSCs). Since no exogenous differentiation factors, such as retinoic acid and brain-derived neurotrophic factor (BDNF) were added to hNSCcm, the data support the conclusion that soluble factors released from cultured proliferative hNSCs altered the cell fate decisions of hMeSCs. It appears that the neural lineage change of hMeSCs is permanent since the daughter cells remained stable for proliferation and neural differentiation after over 30 generations.

### Example 4

### Neuronal Cells Differentiated from Mesenchymal-Derived Neural Stem Cells Exhibited Calcium Activity to Challenges with Potassium Chloride and Glutamate

Neuronal cells derived from the mesenchymal-derived neural stem cells of the invention were assayed for calcium activity in response to potassium chloride and glutamate challenges. Spheres of mesenchymal-derived neural stem cells were transferred to a glass coverslip immersed in a basal medium supplemented with B27 or N2 in a 35 mm Petri dish. Calcium influx responses of these cells to challenges with KCl and glutamate were recorded using a calcium imaging system during the course of differentiation. Cytosolic Ca²⁺ was monitored with ratiometric Ca²⁺ indicator, Fura-2. Coverslips with differentiated cells were washed twice in Hank's Balanced Salt Solution (HBSS, 137 mM NaCl, 5.4 mM KCl, 0.25 mM Na₂HPO₄, 0.44 mM KH₂PO₄, 1.3 mM CaCl₂, 110 mM MgSO₄ and 4.2 mM NaHCO₃) before incubated in 2 ml HBSS containing the fluorescent Ca²⁺ probe Fura-2 AM (5.0 μM) and 0.066% Pluronic F-127. After incubation for 30 to 45 min at 37° C. in darkness, cultures were washed twice in HBSS to remove extracellular dye. Coverslips containing Fura-loaded cells were then mounted to a bath imaging chamber containing Mg²⁺-free HBSS. Fluorescence measurements on individual cells were obtained at excitation wavelengths of 340 nm and 380 nm and emission at 510 nm using an inverted fluorescence microscope (Olympus IX-70). Baseline intracellular Ca²⁺ levels were measured as the ratio of 340/380 nm fluorescence in individual cells incubated in Mg²⁺-free HBSS buffer. Cells were then exposed to 55 mM KCl or 100 μM glutamate. Changes in amplitude of intracellular Ca²⁺ transient level were determined for each cell by subtracting the baseline level of Ca²⁺.

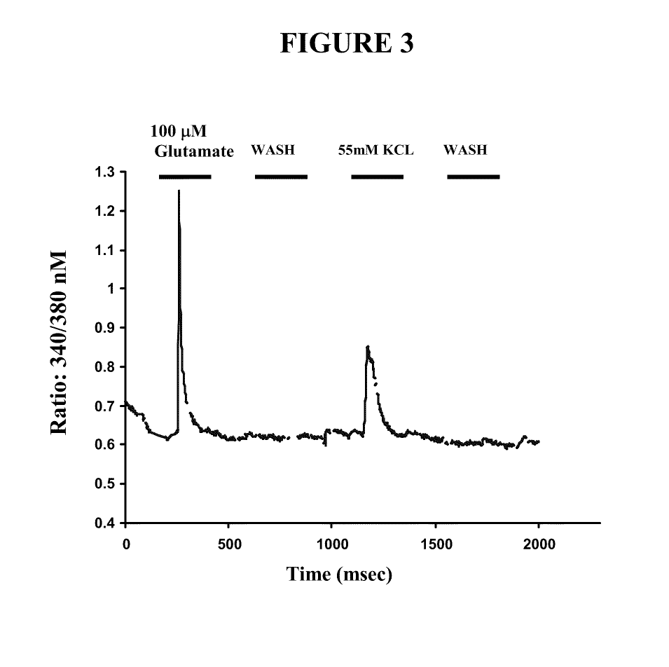

FIG. 3 shows a representative graph of time vs. Ca²⁺ traces illustrating the effect of glutamate and KCl on bone marrow-derived neuronal cells at day six after differentiation. Glutamate produced transient rises in Ca²⁺ that began within seconds after the addition of glutamate and returned to stable states (FIG. 3). The amplitude of Ca²⁺ signal in this experiment after the glutamate challenge was approximately two-fold above baseline. This two-fold increase is representative of differentiated cells examined. A transient rise in Ca²⁺ was also observed in response to KCl (FIG. 3). The results revealed that the calcium activity of bone marrow-derived neuronal cells of this invention in response to challenges with potassium chloride (55 mM) and glutamate (100 μM) is similar to that of CNS-derived neuronal cells in response to the same challenges. The results suggested that the mesenchymal-derived neural stem cells-derived neuronal cells of the invention function similarly to CNS-derived neuronal cells at physiological levels.

### Example 5

### Mesenchymal-Derived Neural Stem Cells Differentiated into Neural Cells Exhibited Membrane Potentials Similar to Neuronal Cells Differentiated from CNS-Derived hNSCs

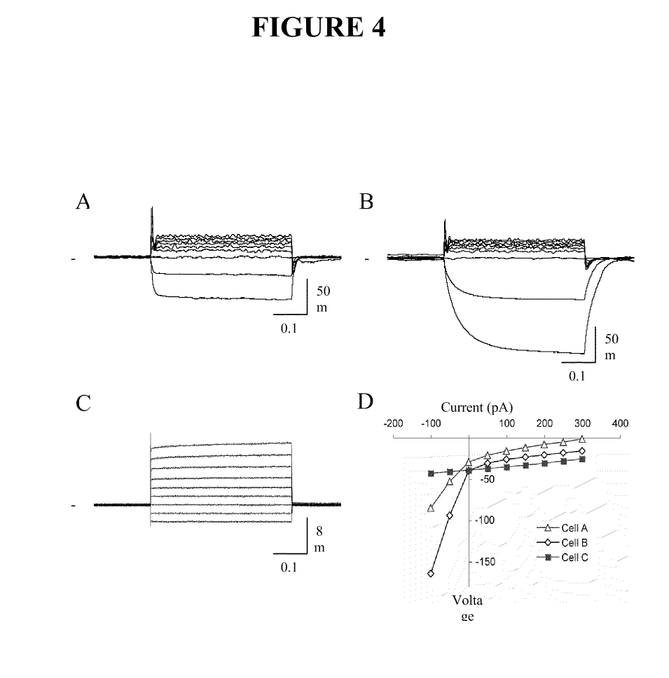

Bone marrow mesenchymal-derived neural stem cells differentiated in vitro into neurons and demonstrated neuron-like membrane properties. FIGS. 4A through 4C show patch-clamp recordings of the responses of two differentiated cells (FIGS. 4A and 4B) and one undifferentiated cell (FIG. 4C) to a series of current pulses. The cells in FIGS. 4A and 4B had spike-like events with overshoot and after-hyperpolarizations at the beginning of the current steps, whereas the cell in FIG. 4C did not. The amplitude of the spike-like structures was 65 mV and the half-width was 4.8 ms for the cell in FIG. 4A. The amplitude was lower (50 mV) and the half-width was shorter (4.0 ms) for the cell in FIG. 4B. Cells were stimulated with 400 ms current steps beginning at −100 pA and increasing to 300 pA in 50 pA intervals. Resting membrane potential (mV) is indicated to the right of the voltage recordings. FIG. 4D shows a plot of the steady-state current-voltage relationship for the cells in FIGS. 4A-4C. Plots were rectifying (non-linear) for the cells in FIGS. 4A and 4B indicating an active voltage response. The cell in FIG. 4C only had a passive (linear) response.

### Example 6

### Transplantation of hNSC or Mesenchymal-Derived Neural Stem Cells into Rodent CNS

Transplantation of hNSCs into aged rodent brain has been shown to improve cognitive function of the animal. Human NSCs, expanded without differentiation in the presence of mitogenic growth factors in serum-free media and labeled with BrdU were injected into the lateral ventricle of mature (6-month-old) and aged (24-month-old) rats (Qu et al., 2001, NeuroReport 12: 1121-1132). The results showed that cognitive function was significantly improved in both mature and aged memory-impaired animals after hNSC transplantation. The results further demonstrated that transplanted clusters of hNSCs into the lateral ventricle of nucleus basalis magnocellularis (NBM) and septum lesion differentiated into GFAP-expressing neural cells. One month after transplantation, transplanted hNSCs were detected in the lesioned areas and expressed mature neuronal marker NeuN immunoreactivity. See (Qu et al., 35th Annual Meeting in Washington, D.C., November, 2005).

Mesenchymal-derived neural stem cells of this invention were tested for their ability to differentiate into neural cells in vivo by transplanting the cells into CNS of normal mice. Mesenchymal-derived neural stem cells of the invention were labeled with 1 μM BrdU for 48 hr in vitro before introduced into mice. When transplanted into the lateral ventricle, hippocampus, and striatum of mature mice (C57/black, four months old), migration and differentiation patterns of the mesenchymal-derived neural stem cells were very similar to the results observed with hNSCs transplantation in rats (Kim et al., 2002, Proc Natl Acad Sci USA 99: 4020-4025, Qu et al., 2001, Id., Ma et al., 36^{th} Annual Meeting in Atlanta, Ga., October, 2006). Mesenchymal-derived neural stem cells differentiated into neurons positive for human beta III-tubulin were detected in the bilateral cingular cortex, the pyramidal layer (CA1 and 3) and dentate gyrus of the hippocampus, and the areas near the lateral ventricle at the injection side of the striatum. In this and the following experiments BrdU was used for the purpose of labeling the injected cells and distinguishing the transplanted cells from host cells. The concentration of BrdU used and the time period of BrdU labeling were less than those used in U.S. Patent Application Publication No. 2003/0219898. Co-localization of BrdU staining and neural marker staining indicated differentiation of the injected mesenchymal-derived neural stem cell into neural cells.

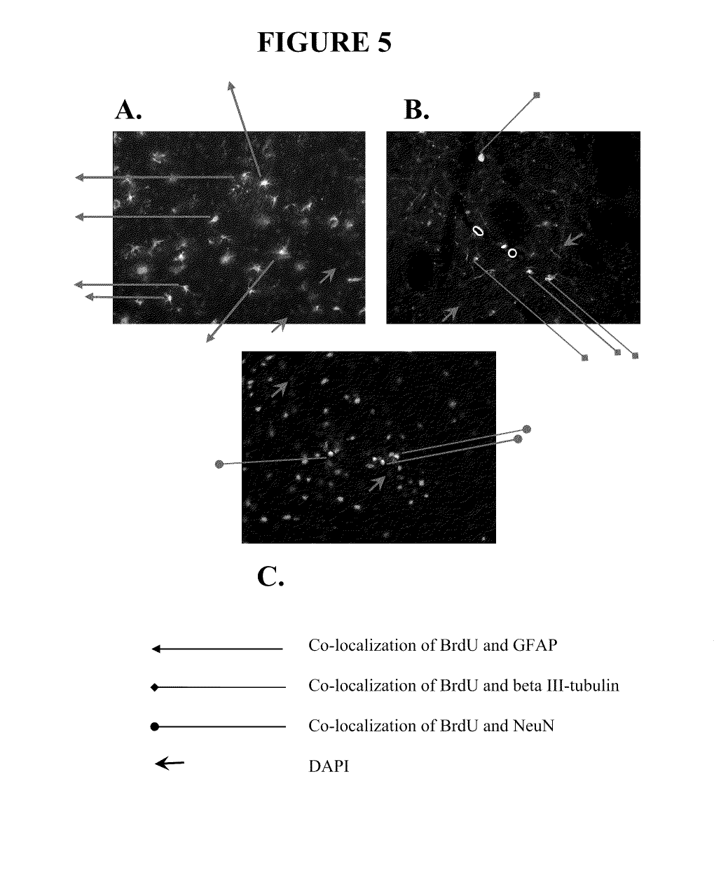

In separate experiments, normal mice injected into the cortex and striatum human mesenchymal-derived neural stem cells of the invention were examined and compared with normal mice injected with human mesenchymal stem cells as control. As shown in FIG. 5A, GFAP-immunopositive cells with BrdU-labeled nuclei in the cortex were detected in brain sections from mice injected with human mesenchymal-derived neural stem cells. BrdU was detected using a sheep anti-BrdU antibody (1:500, Fitzgerald, Concord, Mass.). Transplanted cells differentiated into beta III-tubulin expressing cells that were immunopositive for BrdU were also detected in the striatum near the injection sites in mice injected with human mesenchymal-derived neural stem cells (shown in FIG. 5B). Further, transplanted cells double-stained with BrdU and NeuN were detected in the striatum areas near the lateral ventricle, suggesting that the injected human mesenchymal neural stem cells had migrated away from injection sites and differentiated into cells expressing neuronal marker (shown in FIG. 5C) Exemplary cells that are positive for both BrdU and selective neural markers are indicated in FIGS. 5A-C. No neural differentiation from the transplanted hMeSCs in the control group was detected by immunostaining of GFAP, beta III-tubulin and NeuN (data not shown).

Thus, the in vivo data showed that mesenchymal-derived neural stem cells migrated extensively and differentiated into neurons and astrocytes when transplanted into different regions of mouse brain, including the cerebroventricle, hippocampus, and striatum. The migration and differentiation patterns of mesenchymal-derived neural stem cells in the animal brain were similar to those found in the brains of rats transplanted with hNSC. The results set forth herein demonstrated that mesenchymal-derived neural stem cells differentiated into neural cells similar to NSC-derived neural cells. Strong immune response from host brains was not detected in animals transplanted with the human mesenchymal-derived neural stem cells. Autologous nature of mesenchymal-derived neural stem cells derived from the bone marrow of the same patient can further minimize any undesirable immune response after transplantation.

In addition to immune responses, the safety of the mesenchymal-derived neural stem cells was also assessed. The mesenchymal-derived neural stem cells did not show any abnormal growth and non-neural lineage differentiation up to five months in tissue culture, nor did they show tumor formation in the brain sections examined four weeks after transplantation. During the four-week period post-transplantation, animals showed no signs of ill effects of the transplanted cells. These studies are performed in nude mice for confirmation. Further investigation is conducted to determine whether mesenchymal-derived neural stem cells differentiate into other types of cells after transplantation, which may interfere with brain functions. The neuronal-specific differentiation of mesenchymal-derived neural stem cells is confirmed by the absence of immunocytochemical staining for markers of adipogenic, osteogenic, and chondrogenic lineages, which are the default differentiation pathways for hMeSC. In the group of mice of the animal stroke model as described below (where mesenchymal-derived neural stem cells are introduced via tail vein by intraveneous (i.v.) injection), any immediate reactions, including allergic reactions, in response to the transplantation are examined. Additionally, chromosome numbers in mesenchymal-derived neural stem cells of the invention are examined to confirm that mesenchymal-derived neural stem cells maintain normal karyotypes after long term culturing.

### Example 7

### Effects of Mesenchymal-Derived Neural Stem Cells Transplantation on Mice Suffering from Stroke in a Mouse Stroke Model

Stroke is the third leading cause of death and the leading cause of long-term disability in the United States. With the aging of the population, the number of stroke patients in the U.S. is likely to grow substantially. NSCs existing in brain tend to migrate toward stroke sites; this phenomena has suggested an intrinsic mechanism for brain self repair (Parent, 2003, Neuroscientist. 9(4):261-72, Hoehn et al., 2002, Proc Natl Acad Sci USA. 99(25):16267-72). However, the numbers of endogenous NSCs are small, and transplantation of mesenchymal-derived neural stem cells of the invention may enhance this intrinsic potential and/or directly replace those damaged brain cells.

Although donor hNSCs are potential candidates for neuroreplacement therapy, their clinical use is hampered by the unfortunate prospect of immune rejection elicited by these cells and various other factors as described herein. The approach of using autologous mesenchymal-derived neural stem cells can significantly overcome hurdles associated with embryonic or fetal stem cell-based transplantation.

The therapeutic efficacy of mesenchymal-derived neural stem cells of the invention is tested in an animal stroke model. The middle cerebral artery occlusion (MCAO) model has been used as a validated model for stroke in animals. Although widely used, this model requires surgical procedures and lesions produced are variable in size and location. Recovery after surgery has not been consistent. These factors make the interpretation of the effects of therapeutic interventions difficult.

In rodents, photoactivation by cold white light of intravenously infused rose Bengal (RB, 4,5,6,7-tetrachloro-2′,4′,5′,7′-tetraiodofluorescein) has been shown to produce free radicals, which caused consistent and reproducible cerebral photothrombosis that shared essential inflammatory responses with focal ischemia produced by middle cerebral artery occlusion (Watson et al., 1985, Ann Neurol, 17:497-504, Schroeter et al., 2002, Neurosci Methods, 117:43-49). The histological features of infarcts in surgical and autopsy specimens have indicated that this model is relevant to acute human ischemic infarcts (Lee et al., 1996, Stroke, 27:2110-2119). Following the procedure, RB (10 mg/kg) was injected into the tail vein of three month old male mice under deep anesthesia, followed by exposure of the mice to three minutes of cold white light (0.1 Walts/cm²). The size and location of the lesions were controlled by stereotaxic parameters and light exposure time to produce consistent photochemical lesions in the sensorimotor cortex. The results showed that the brain sections obtained from the animals of the mouse stroke model exhibited consistently a picture of an acute infarct zone, a transition zone, and surrounding normal neurons in the lesioned frontal lobes of the brain. These mice with sensorimotor cortex photothrombosis stroke showed impaired forelimb function: they were not able to hold on to a vertical grid firmly and fell off from the grid immediately. This non-invasive cerebral photothrombosis model of stroke in mice has thus been established and is used for cell transplantation studies.

Mice (eight for each group) with cerebral photothrombosis stroke induced by RB infusion and white light exposure are anesthetized with sodium pentobarbital (50 mg/kg i.p.). Mesenchymal-derived neural stem cells of the invention are labeled with 1 μg (1 μM) BrdU for 48 hr in vitro before introduced by intravenous infusion, cerebroventricle delivery or peri-lesional injections. Specifically, the pre-labeled cells are injected into: (1) areas surrounding the infarct zone (Group I), (2) the lateral ventricle (LV) at the lesioned side (Group II), and (3) the tail vein in the stroke model mice via i.v. injection (Group III) at two weeks post-stroke to avoid inducing an acute phase of inflammation immediately following stroke. Control animals include sham (stroke animals with vehicle injection only) and placebo (stroke animals with naïve hMeSC injection). Two different doses of mesenchymal-derived neural stem cells (1×10⁴ and 1×10⁶ cells) are injected into the eight animals in each treatment group.

Immune suppression is not used for mesenchymal-derived neural stem cell transplantation because, as shown in earlier studies, mesenchymal-derived neural stem cells did not evoke strong immune responses when transplanted into normal mouse brain. Given that the invention utilizes an autologous approach, use of immune suppression is not likely necessary.

All animals in each group are tested for motor function by measuring the time period within which they are able to hold on to a vertical grid after the stroke. Baseline motor function assessment is established with three independent readings per animal repeated at least twice. Animal motor function is determined one and two weeks after stroke, and two and four weeks after cell transplantation. At each time point of assessment at least three independent readings per animal is recorded. Animals are sacrificed at eight weeks post-transplantation by an overdose of anesthesia. Additional groups are allowed to survival for 24 weeks after cell transplantation and sacrificed for long term safety of cell transplantation studies. Brain sections are prepared for histology and stained for assessment of size of infarct. Immunohistochemistry is performed for survival, migration, and differentiation of transplanted mesenchymal-derived neural stem cells. Antibodies specifically recognizing human protein are used to identify the transplanted mesenchymal-derived neural stem cells. Antibody specific for BrdU is used as the second marker for the transplanted cells. Since BrdU can be released from cellular DNA from dead transplanted cells, and subsequently absorbed by host proliferating cells, double-fluorescent staining with antibodies recognizing human nuclei protein (CHEMICON) and BrdU is used to identify the transplanted cells. The number and percentage of transplanted cells showing particular antibody markers in the areas of interest are determined. Synapse formation between mesenchymal-derived neural stem cells and host brain cells is examined electromicroscopically as well as analyzed by using specific antibodies against human-specific synaptophysin or other synapse markers. The histological and immunohistochemical examination and behavioral testing results for the mesenchymal-derived neural stem cell transplantation are compared to corresponding groups receiving naïve hMeSC and sham transplantations. The data are analyzed by analysis of variance (ANOVA) for all pairwise comparisons, followed by post-hoc analysis, such as Fisher's least significant difference (LSD) analysis. Digitally-captured images from fluorescent microscopy of cultured cells are analyzed by NIH Image software with Cell Scoring, Particle Analysis, and Cell Analysis macros. Further histological examinations for any abnormal cell growth and tumorogenicity are performed in tissue sections from multiple organs including heart, liver, lung, spleen, and thymus to determine whether mesenchymal-derived neural stem cells would migrate through the lesioned blood brain barrier (BBB) into other organs.

It should be understood that the foregoing disclosure emphasizes certain specific embodiments of the invention and that all modifications or alternatives equivalent thereto are within the spirit and scope of the invention as set forth in the appended claims.

What is claimed is:

1. A method for making mesenchymal-derived neural stem cells from a mammal's bone marrow, the method comprising:
(a) isolating mesenchymal stem cells from the bone marrow; and
(b) culturing the mesenchymal stem cells in a cell-free human neural stem cell conditioned medium to produce the mesenchymal-derived neural stem cells,
wherein the mesenchymal-derived neural stem cells are capable of differentiating into neural cells.

2. The method of claim 1 wherein the mammal is a human.

3. The method of claim 2 wherein the human has a neurological disease, deficit, defect, deficiency or disorder.

4. The method of claim 3 wherein the neurological disease, deficit, defect, deficiency or disorder is traumatic brain injury, spinal cord injury, Parkinson's disease, Alzheimer's disease, amyotrophic lateral sclerosis, motor neuron diseases, stroke or sequelae of stroke.

5. The method of claim 4, wherein the neurological disease, deficit, defect, deficiency or disorder is stroke.

6. The method of claim 1 wherein the bone marrow is fresh or frozen bone marrow.

7. The method of claim 1, wherein the cell-free human neural stem cell conditioned medium is obtained by a process comprising the steps of culturing human neural stem cells in serum-free medium containing B27 or N2 in the presence of bFGF (basic fibroblast growth factor) and EGF (epidermal growth factor) and optionally LIF (leukemia inhibitory factor), and collecting the cell-free conditioned medium after at least 2-3 days in culture.

8. The method of claim 7, wherein the cell-free conditioned medium is collected every 2-3 days of the culture and the human neural stem cells are cultured for no more than 60 days.

9. The method of claim 7 wherein the serum-free medium contains B27.

10. The method of claim 7 wherein the mesenchymal stem cells are cultured in the cell-free human neural stem cell conditioned medium in the presence of at least one mitogenic growth factor.

11. The method of claim 10 wherein the mitogenic growth factor is bFGF, EGF, LIF (leukemia inhibitory factor), PDGF (platelet-derived growth factor) or FGF8.

12. The method of claim 1 wherein the mesenchymal-derived neural stem cells express early neural marker SSEA4, BF1, Nurr1, NeuroD, OCT4, Nanog, POU domain transcription factor 4 (OCT4), Nanog, human paired box gene 6 (Pax6), Sox1, or Nestin.

13. Isolated mesenchymal-derived neural stem cells produced by the method according to claim 1.

14. The mesenchymal-derived neural stem cells of claim 13, wherein the mammal is a human.

15. The mesenchymal-derived neural stem cells of claim 14 wherein the human has a neurological disease, deficit, defect, deficiency or disorder.

16. The mesenchymal-derived neural stem cells of claim 15 wherein the neurological disease, deficit, defect, deficiency or disorder is traumatic brain injury, spinal cord injury, Parkinson's disease, Alzheimer's disease, amyotrophic lateral sclerosis, motor neuron diseases, stroke or sequelae of stroke.

17. The mesenchymal-derived neural stem cells of claim 16, wherein the neurological disease, deficit, defect, deficiency or disorder is stroke.

18. The mesenchymal-derived neural stem cells of claim 13 wherein the bone marrow is fresh or frozen bone marrow.

19. The mesenchymal-derived neural stem cells of claim 13, wherein the mesenchymal-derived neural stem cells are capable of forming a neurosphere.

20. The mesenchymal-derived neural stem cells of claim 13, wherein the cell-free human neural stem cell conditioned medium is obtained by a process comprising the steps of culturing human neural stem cells in serum-free medium containing B27 or N2 in the presence of bFGF (basic fibroblast growth factor) and EGF (epidermal growth factor) and optionally LIF (leukemia inhibitory factor), and collecting the cell-free conditioned medium after at least 2-3 days in culture.

21. The mesenchymal-derived neural stem cells of claim 20, wherein the cell-free conditioned medium is collected every 2-3 days of the culture and the human neural stem cells are cultured for no more than 60 days.

22. The mesenchymal-derived neural stem cells of claim 20 wherein the serum-free medium contains B27.

23. The mesenchymal-derived neural stem cells of claim 22 wherein the mesenchymal stem cells are cultured in the cell-free human neural stem cell conditioned medium in the presence of at least one mitogenic growth factor.

24. The mesenchymal-derived neural stem cells of claim 23, wherein the mitogenic growth factor is bFGF, EGF, LIF (leukemia inhibitory factor), PDGF (platelet-derived growth factor) or FGF8.

25. The mesenchymal-derived neural stem cells of claim 13 wherein the mesenchymal-derived neural stem cells express early neural marker SSEA4, BF 1, Nurr1, NeuroD, OCT4, Nanog, OCT4, Nanog, Pax6, Sox1, or Nestin.

26. The mesenchymal-derived neural stem cells of claim 13 wherein the mesenchymal-derived neural stem cells do not exhibit action membrane potential of mature neurons.

27. The mesenchymal-derived neural stem cells of claim 13 wherein the mesenchymal-derived neural stem cells are committed to neural lineage in differentiation.

28. The mesenchymal-derived neural stem cells of claim 27 wherein the mesenchymal-derived neural stem cells are capable of differentiating into neural cells in a basal medium.

29. The mesenchymal-derived neural stem cells of claim 28 wherein the basal medium is further supplemented with B27 or N2.

30. The mesenchymal-derived neural stem cell of claim 29, wherein the basal medium is further supplemented with B27.

31. The mesenchymal-derived neural stem cells of claim 30 wherein the basal medium further comprises at least one neurotrophic factor neural growth factor (NGF), brain-derived neurotrophic factor (BDNF), or glial cell line-derived neurotrophic factor (GDNF).

32. The mesenchymal-derived neural stem cells of claim 13 wherein the mesenchymal-derived neural stem cells are capable of differentiating into neural cells when administered into a patient's central nervous system (CNS).

33. The mesenchymal-derived neural stem cells of claim 13 wherein the mesenchymal-derived neural stem cells are capable of differentiating into neural cells that express GFAP, NG2, beta III tubulin, MAP2, or NeuN, or are immunopositive for GalC or GABA.

34. A pharmaceutical composition comprising the isolated mesenchymal-derived neural stem cells of claim 13, and at least one pharmaceutically acceptable excipient, diluent or carrier.

35. A method for treating a patient having a neurological disease, deficit, defect, deficiency or disorder, the method comprising administering the mesenchymal-derived neural stem cells of claim 13 to the patient, wherein the mesenchymal-derived neural stem cells differentiate to neural cells in a tissue-specific manner that reduces the neurological disease, deficit, defect, deficiency or disorder in the patient.

36. The method of claim 35, wherein the patient is a human.

37. The method of claim 36, wherein the mesenchymal-derived neural stem cells are derived from mesenchymal stem cells isolated from the patient's bone marrow.

38. The method of claim 37, wherein the neurological disease, deficit, defect, deficiency or disorder is traumatic brain injury, spinal cord injury, Parkinson's disease, Alzheimer's disease, amyotrophic lateral sclerosis, motor neuron diseases, stroke or sequelae of stroke.

39. The method of claim 38, wherein the neurological disease, deficit, defect, deficiency or disorder is stroke.

40. The method of claim 39, wherein the patient suffers from tissue damage in the neural tissue.

41. The method of claim 40, wherein the mesenchymal-derived neural stem cells migrate to the area of tissue damage, differentiate in a tissue-specific manner at the area of tissue damage, and function in a manner that ameliorate the neurological disease, deficit, defect, deficiency or disorder.

42. The method of claim 35, wherein the patient is administered with the mesenchymal-derived neural stem cells and a pharmaceutically acceptable carrier, diluent or excipient.

43. A method for treating a patient having a neurological disease, deficit, defect, deficiency or disorder, the method comprising administering the pharmaceutical composition of claim 34 to the patient, wherein the mesenchymal-derived neural stem cells of the pharmaceutical composition differentiate to neural cells in a tissue-specific manner that reduces the neurological disease, deficit, defect, deficiency or disorder in the patient.

44. The method of claim 43, wherein the mesenchymal-derived neural stem cells are derived from mesenchymal stem cells isolated from the patient's bone marrow.

45. A cell-free human neural stem cell conditioned medium, said conditioned medium is obtained by a process comprising the steps of culturing human neural stem cells in a serum-free medium containing B27 or N2 in the presence of bFGF and EGF and optionally LIF, and collecting the cell-free conditioned medium after at least 2-3 days in culture.

46. The cell-free human neural stem cell conditioned medium of claim 45, wherein the cell-free conditioned medium is collected every 2-3 days of the culture and the human neural stem cells are cultured for no more than 60 days.

47. A method of preparing a cell-free human neural stem cell conditioned medium of claim 45.

48. The method of claim 47, wherein the human neural stem cells are cultured in the serum-free medium that further comprises LIF (leukemia inhibitory factor).

49. A kit for making mesenchymal-derived neural stem cells, said kit comprising a cell-free human neural stem cell conditioned medium of claim 45 and instructions for use thereof.

50. The kit of claim 49, further comprising at least one mitogenic growth factor.

51. The kit of claim 50, wherein the mitogenic growth factor is bFGF, EGF, LIF, PDGF or FGF8.

52. The kit of claim 51 further comprising reagents for the preparation of Ficoll-Hypaque gradients for isolating mesenchymal stem cells from bone marrow.
